(19) United States
(12) Patent Application Publication
(10) Pub. No.: US 2021/0379425 A1
(43) Pub. Date: Dec. 9, 2021

(54) PERSONAL PROTECTION AND MONITORING

(71) Applicant: Bich Q. Tran, Saratoga, CA (US)

(72) Inventor: Bich Q. Tran, Saratoga, CA (US)

(21) Appl. No.: 16/894,040

(22) Filed: Jun. 5, 2020

(51) Int. Cl. A62B 23/06

(52) U.S. Cl. CPC A62B 23/06; A62B 9/006

(57) ABSTRACT

A personal protection device includes one or two elongate members for engaging into the nostrils of the nose of a user. The elongate members each includes gridded ports for allowing air to pass through the elongate member when breathing in and for allowing air to exit via the elongate member when breathing out. Sensors are placed in the air flow pathway to detect pathogen presence, and the primary air filter is positioned in the elongate member. In yet other embodiments, a disposable version is disclosed. The eyewear can also be used as part of an extended reality system, and when game playing, particularized scents can be rendered to the nose to enhance the gaming experience. In another aspect, systems and methods protect against pathogen by sampling an environment of a travel path with a plurality of pathogen detectors along the travel path to detect a presence of one or more pathogens, wherein at least one detector includes a nano-sensor with receptacles to bind to the pathogens and wherein the nano-sensor changes resistivity, inductance or capacitance upon pathogen binding; directing air towards said pathogen detectors; contact tracing a user mobile device having a mobile identification (ID) carried by each user, wherein the mobile device comprises a memory storing mobile IDs of all devices within a predetermined radius of the user mobile device; and performing deep learning with a neural network receiving data from the pathogen detectors and to the user mobile device to detect a presence of one or more pathogens.

Coronaviruses (CoVs) constitute a group of phylogenetically diverse enveloped viruses that encode the largest plus strand RNA genomes and replicate efficiently in most mammals. Human CoV (HCoVs-229E, OC43, NL63, and HKU1) infections typically result in mild to severe upper and lower respiratory tract disease. Recently, COVID-19 emerged in China in December 2019 with symptoms including cough, fever, shortness of breath, muscle aches, sore throat, unexplained loss of taste or smell, diarrhea and headache. COVID-19 can be spread from person to person and may be spread through droplets released into the air when an infected person coughs or sneezes. The droplets generally do not travel more than a few feet, and they fall to the ground (or onto surfaces) in a few seconds—this is why social and physical distancing is effective in preventing the spread. The droplets can be larger than 5 μm, and it can also be less than 5 μm (nuclei droplet)]. The virus can also be attached to fine particles in the respiratory track or mouth of the infected person and got aerosolized during coughing, sneezing, speech and vomiting. The aerosolization of virus may be one of the several causes that explains the sustained person-to-person spread of the COVID-19 in the world in such a short period of time.

The size of the COVID-19 has been determined under Transmission Electron Microscope (TEM) to be 60-140 nm, which averages to 100 nm]. This is similar in size as the SARS coronavirus, which is also 100 nm. The common influenza virus is 80-120 nm, which average out to 100 nm. As the virus can be attached to particulates less than 100 nm, the smallest size for the COVID-19 and its carrier (droplet or particle) can still be about 100 nm. The 100 nm aerosol has been referred to as nano-aerosol, nanoparticle or ultrafine particle. While the National Institute for Occupational Safety and Health (NIOSH) has standardized N95 and N98 respirator at 300 nm, there is no standard filtration test for nano-aerosols at 100 nm.

### SUMMARY

A personal protective equipment (PPE) includes a wearable housing to distribute air to a nostril; an air filter to filter the air; and a mount to secure the wearable housing to a nose.

Advantages of the system may include one or more of the following. The PPE is inconspicuous, unlike the present masks. The PPE is smart and provides sensors to monitor the user. The sensors can detect pathogens, or can detect vital signs for health monitoring. The PPE can use edge processing and 5G low latency to offload processing requests to remote computers and only render for the user, thus reducing power consumption.

### BRIEF DESCRIPTION OF THE DRAWINGS

The above-mentioned features will become more clearly understood from the following detailed description read together with the drawings in which:

FIG. 1A-1B are exemplary views of a wearable nasal PPE device.

FIG. 2A shows an exemplary eye ware and nose cover combination that provides filtered air to the nose and visual functions with the eyeglasses.

FIG. 2B shows an exemplary flexible electronic or sensor.

FIG. 2C shows an exemplary eye wear embodiment with a mouth and nose protection.

FIG. 3A-3B show exemplary views of a disposable nostril nasal PPE device.

FIGS. 4A-4B shows an exemplary walkway with sensors to detect population health.

FIG. 4C shows an exemplary contact tracing module.

FIG. 4D shows an exemplary CT security scanner.

FIG. 4E shows an exemplary lung image.

### DESCRIPTION

Various embodiments show exemplary personal protective equipment (PPE) devices that are inconspicuous, yet effective in preventing pathogens from entering a body.

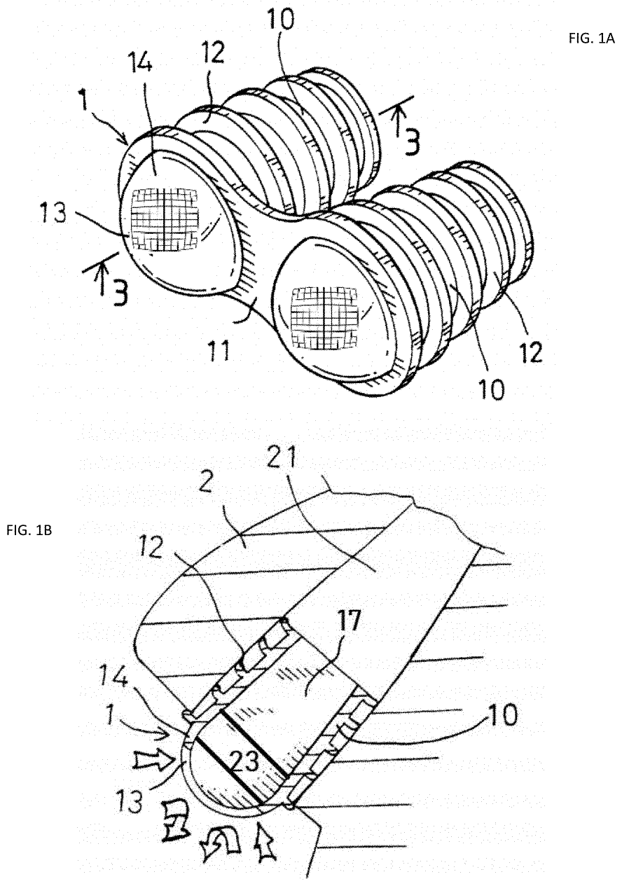

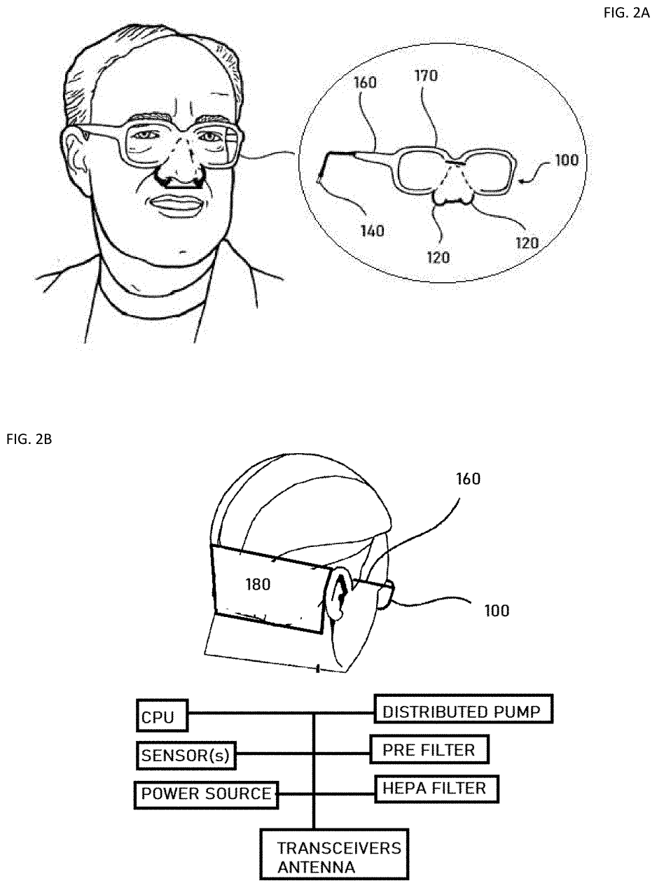

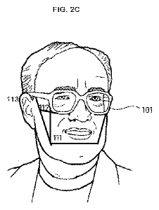

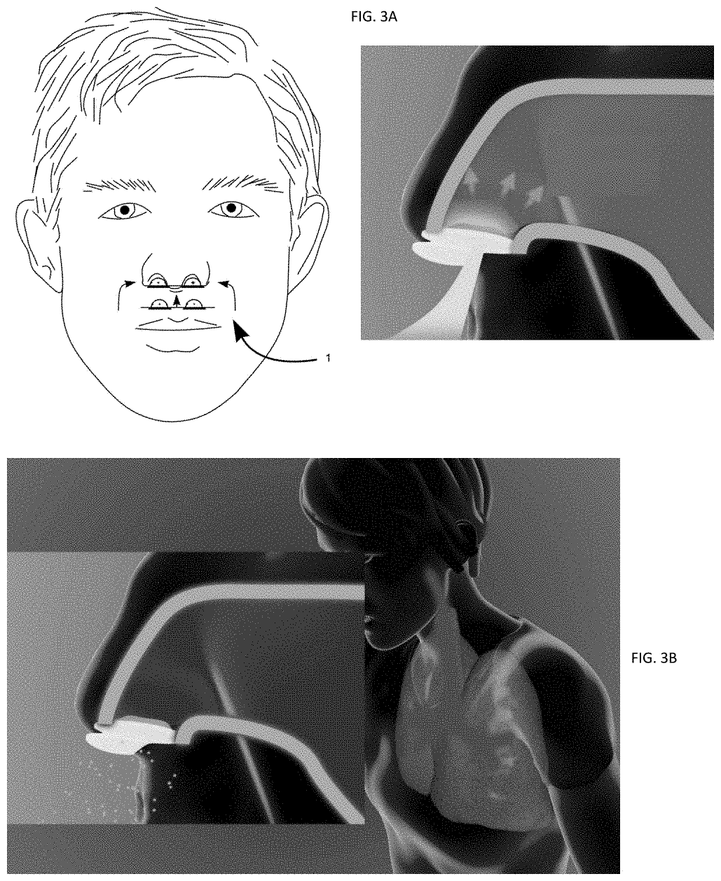

FIG. 1A-1B are exemplary views of a wearable nasal PPE device, while FIG. 2A shows an exemplary eyeware/nose cover combination that provides filtered air to the nose and visual functions with the eyeglasses. FIG. 2B shows exemplary electronics that can be used in FIG. 1A or 2B. The electronics of FIG. 2B are optional for FIG. 1A or 2A. FIG. 3A-3B show exemplary views of a low cost disposable nostril nasal PPE device. The device of FIGS. 3A-3B can be with or without electronics.

The personal protective equipment (PPE) 1 includes a nose plug device provided for engaging into a nose of the users (FIG. 1). For example, the nose plug comprises two elongated nostril members 10 for engaging into the nostrils of the nose. The elongated nostril members 10 each preferably includes one or more peripheral flanges 12 extended radially outward therefrom for engaging with the nose and for making an airtight seal with the nose. A connector 11 is provided between the front portions of the elongate members 10, for linking the elongate members 10 together, and for preventing the nostril members 10 from being deeply engaged into the nose.

The elongate members 10 of the nose plug device 1 each includes a chamber 13 formed therein, and each includes an optional valve 14 provided therein, such as provided in the front portion thereof. For example, the device has a convex or outwardly or forwardly curved membrane structure having grids or slits 13 formed therein, particularly formed in the middle portion thereof. The elongate nostril member 10, particularly the peripheral flanges 12 of the elongate members 10 and/or the slits/grids 14 are preferably made of silicone, rubber, gel materials or the like that includes a suitable resilience for clamping the elongate members 10 within the nose nostrils 21, and for allowing the grids/slits 13 and the optional valves 14 to be opened when air flows out through the valves 14 and to be closed when air is going to flow into the nose so that incoming air has to go through the filter 17 before reaching user lungs. In this manner, air being exhausted by breathing out is not filtered, while incoming air is filtered.

In other embodiments, air breathed in and out is filtered to protect the user and nearby people for physical distancing needs. Because the coronavirus can travel on liquid droplets breathed or coughed out by infected people, an array of health authorities recommends staying away from crowds and maintaining physical separation from others. It's why restaurants, bars, stores, and other places where people mingle closely have faced such economically devastating restrictions. The U.S. Centers for Disease Control and Prevention (CDC) specifically recommends a six-foot buffer. A three-foot guideline can be used for exhaled droplets when protected by the present PPE.

A multi-level air filter 17 is provided in the nostril members 10. The filter can be washable or can be user replaceable. In one embodiment where electrostatic power is used to capture pathogen, the filter does not need washing or regular replacement.

One embodiment provides an electrostatic filter to capture aerosols with pathogens therein. By rearranging nanofibers into multiple modules (multi-modules), or multiple layers (in short multilayers), in the filter, macropores of the scrim material can be introduced into the filter disrupting an otherwise two-dimensional nanofiber mat dominated by micropores. This increased the thickness of the filter drives the pressure drop. By rearranging the thick nanofiber mat into multilayers with each layer supported by a scrim porous material, the latter also served as a barrier to reduce the electrical interference between adjacent nanofiber layers in the filter on challenging aerosols, thereby improving capture of nano-aerosols by electrostatic effect. With the aerosol capture mechanisms, diffusion works well for aerosols less than approximately 100 nm, interception for aerosols greater than approximately 100 nm and less than 1 μm, and interception for aerosols larger than 1 μm, it is advantageous to have small fiber diameters that offer large specific surface to enhance these mechanisms.

Another filter made of nanofibers with diameter less than 1 μm with large specific surface can be used. Small amounts of nanofibers are required, on the order of less than 1 gram of fiber per square meter (gsm) filter area. To reduce pressure drop the same amounts of nanofibers to achieve a certain efficiency are put into multiple layers (or multiple modules) with each thin nanofiber layer laid on a permeable substrate (a module) with large permeable macropores inter-dispersed in the filter stack. The multiple layers (hereafter referred as multilayer) have nearly similar filtration efficiency as a single layer, yet the pressure drop is significantly reduced.

To improve the filter efficiency without incurring pressure drop the nanofibers can carry electrostatic charges. A polyvinylidene fluoride (PVDF) nanofiber mat can be fabricated using corona discharge. The electrostatic charges can stay for 3 months with only 1% reduction in filtration efficiency. This is due to using corona discharge charging the nanofibers by implanting more stable space charges in the nanofiber mat. A stronger electric field can greatly affect dielectrophoresis, i.e. first inducing dipoles on approaching neutrally charged aerosols and subsequently attracting the charged aerosol by the charged fiber by interacting with the opposite charge of the dipole on the particle.

Sensor(s) 23 can be positioned in, before, or after the filter 17. In operation, the slits 13 of the check valves 14 of the nose plug device may be opened when the users breathe the air or force the air to flow out of the elongate nostril members 10. When the user is not breathing or does not force the air to flow out of the elongate members 10, the check valves 14 may be closed, for preventing the air from flowing into the nose of the user from outer environment. When the user breathes in, air goes through the air filters to keep external virus/pathogen out of the nose/lung/body. When the user breathes out, air goes through the filter before air is released back to the environment. The sensors detect pathogens and health conditions for notifying the user or for subsequent treatment.

Yet another embodiment provides Airborne Virus Capture and Inactivation by an Electrostatic Particle Collector. An electrostatic precipitator (ESP) provides specific electric field strength at a given applied voltage. The ESP can be a cylindrical tube with a wire along its axis or can be a parallel plate ESP. Unlike in a parallel-plate ESP, where the electric field is uniform in the region between the plates, the electrical field strength inside a cylindrical ESP varies as a function of radial position.

FIG. 2A shows an exemplary eyeware/nose cover combination that provides filtered air to the sealed environment of the nose and visual functions with the eyeglasses. FIG. 2B shows an exemplary flexible electronic or sensor. The air filtration system can be concealed with the eyeglasses on the person and does not interfere with the user's normal activities such as speaking, dining, traveling, etc. The user breaths normally through the nose without any restrictions. The user can also perform computing functions using glass-mounted displays for extended reality purposes. Moreover, for VR gaming or AR processing, 5G edge processing can be done using the cellular transceiver connected to the eyeglasses. In one embodiment, the eyeglasses 100 are fitted over the user's eyes, and the eyeglasses have an integral transparent bridge portion between the two glasses that is fitted over the nose. Gravity causes the eyeglasses with integral nose cover to be secured against the nostril openings, thus securing the air inlet to the lung with filtered air delivered on the frame of the eye-glasses. Thus, filtered air at positive pressure is delivered directly to the nose in a non-conspicuous manner. While the nose protection is detailed herein, mouth protection is also contemplated. In another embodiment, the nose bridge portion 120 is expanded to cover the mouth as well to provide additional PPE protection.

FIG. 2A shows the preferred method of inconspicuously delivering filtered air to the user's nose. Hollow eyeglass frames 100 deliver filtered air from a hose behind the head to nose inlets/tubes 120. Nose inlet 120 can be small air inlets or tubes that direct filtered air from the hollow eyeglass frame rims up into the nostrils. These tubes will be molded and colored such that they closely follow and blend in with the contour of the user's face between the frames and the nostrils.

In operation, filtered compressed air is forced through the frames and nose tubes at a flow rate greater than the user's normal peak inhalation rate. That is, the flow rate through the nose tubes is adjusted to be high enough so that some excess filtered air is being exhaled out the nose during normal inhalation. This exhaled filtered air prevents unfiltered outside air from entering the nostrils during inhalation. During user exhalation, all the filtered air will be exhaled as well.

The hollow eyeglass frames and nose tubes form the heart of the preferred embodiment of the present invention and will be offered in a variety of contemporary styles. Since the system is a positive pressure powered system, there is no respiratory stress to the user. Since the mouth is not covered in the embodiment of FIG. 2A (but contemplated), the system does not interfere with normal conversation. IF the mouth is covered, the AR/VR/XR components allow the user to communicate by texting or typing, or use of EEG to select brief messages, among others.

The hollow eyeglass frames and nose tubes may also be useful to cannabis/CBD/CBG or oxygen therapy patients that desire an unobtrusive means of oxygen delivery when at work or out and about.

The eyeglass frame can provide air passageways to guide filtered air to the nose in one embodiment, and to both nose and mouth in another embodiment. A respirable air hose connection to the eyeglass frames 100 can be used. The hoses can be contoured around the user body to be inconspicuous. An air compressor or suitable fans and blowers can provide the desired flow rates through the air distribution ducts formed into the frames. A linear actuator can be used as a piston to provide air compression in a compact manner. Upon actuation, the piston can be made to alternately move back and forth inside an air tube. Multiple actuators can be controlled to act as a distributed pump, and pump noise and vibration should be minimal because pistons in adjacent compression tubes are moving in opposite directions which will cause the piston momentum forces in adjacent tubes to cancel.

FIG. 2B illustrates the had mounted air filter and control electronics along with sensors. Outside air is drawn in through the prefilter by the distributed pump and then forced through HEPA filter, among others. Battery pack supplies power to the pump. In addition to filter meshes, activated carbon granules or other adsorbent may be added to the inlet side of HEPA filter module for absorbing odors and for eliminating ozone. Another effective absorbent is cpz which is a mixture of carbon, permanganate, and zeolite. In another embodiment, germicidal lamps or ultraviolet lamps can radiate at 253.7 nm wavelength to sterilize bacteria, viruses, yeasts and molds.

A prefilter can filter all large dust particles out of the input air so as to protect the pump. Typically, low or moderate efficiency air filters are used for this purpose to reduce filter air flow restriction when fans or blowers are used. Since an air compressor is used in the present invention, filter air flow restriction is not as great a problem. An optional moisturizer module may be useful in dry conditions to keep from drying out the nose tissues. The air cooler module may be required to remove germicidal lamp heat from the air stream when using the sterilization module. It will be constructed using solid state thermoelectric cooler devices. The heater module will be constructed using a resistive heating element. The heater and cooler may be useful in either extremely cold or hot environments respectively or for asthma patients who cannot tolerate rapid air temperature variations. The heater and cooler modules will be thermo-statically controlled to automatically maintain the temperature selected by the user.

In another embodiment, a UV light with a protective cover to prevent biological cell damage is used to treat incoming air. The UV light can be delivered by fiber optic cables to air passageways to inactivate a virus or pathogen at an inlet point. Additionally, the filter includes specific fibers or fiber coatings that collect moisture from exhaled air and delivers moisture to air inhaled into the nostrils during normal breathing.

FIG. 2C shows an embodiment with a mouth and nose protection. In this embodiment, a mouth mask or wearable housing 111 extends from the eye wear device 101. The eye wear device 101 also has curtains or masks 112 to enclose the sides. In one embodiment, the wearable housing 111 includes curtains for air seal, or masks 111/112 which can be rigid, but they can also be flexible to make it easy to flip the curtains to the top, the bottom, or to the sides as desired. Sensor(s) 113 on housing 111 proximal to the ear when worn can detect vital signs. The curtain/masks can be permanently secured to the frame 101 by glue or screw, or can be non-permanent attachment via magnet mounting to the frame, for example. To protect the user against leakage, a positive air pressure is maintained in the volume immediately in front of the nose or mouth so that virus in the environment cannot enter the nose/mouth.

FIG. 3A-3B show exemplary disposable embodiments with a FIG. 8 fabric pad/base acting as a particulate filter, where the other side of the particulate filter has an adhesive side and one or more air filter domes projecting therefrom. The domes contain a primary air filter with fibers nano-dimensioned to filter or keep out coronaviruses. The domes can also contain compositions that kill remaining viruses that managed to pass through the front-end particulate filter. For example, a humectant substance coated onto the fabric pad material is utilized to collect moisture and keep the fabric material moist. Specifically, a humectant kills the viruses, and further attracts water vapor in the exhaled air by absorption and delivers that moisture to the inhaled air.

In one embodiment, humectant in combination with other compositions, produces a synergistic anti-viral effect when used in combination. The formulation contains a humectant such as sorbitol, glycerol, or other comparable compound in the range of 20-80% w/v final concentration, which facilitates structural and/or functional three dimensional disruption or disorientation of the viral envelope. The activation of the destruction of the envelope and subsequent death of the envelope virus is then achieved using combination of inorganic monovalent anions, nonionic detergents and anionic detergents. These monovalent anions can include sodium bicarbonate, sodium thiocyanate, sodium fluoride and sodium chloride at about 0.5 to 5% w/v final concentration; nonionic detergents such as Tween 20 at concentrations of about 0.1% to 3% v/v, ethanol up to about 15% v/v, and other antimicrobial agents such as chlorhexidine or comparable basic substances at concentrations from about 0.01 to 0.2% v/v. The formulation may also include anionic surfactants, flavor and water added to 100%.

In another embodiment, sodium polyacrylate fibers in the fabric pad are utilized to absorb water vapor into the fabric and release that moisture into the inhaled breath. Sodium polyacrylate has the capability to absorb up to 200 times its mass in water. In other embodiments, other natural, hygroscopic fibers, such as cellulose, cotton, wool and silk are used to absorb moisture from the exhaled breath and deliver that moisture to the inhaled breath. In still other embodiments, other hygroscopic natural plant-based fibers, such as hemp and jute or animal-based hygroscopic fibers, such as mohair and alpaca, or man-made hygroscopic fibers, such as rayon, are used. In various embodiments, humectants, sodium polyacrylate, and other such materials are utilized together to provide for absorbing moisture into the fabric pad or domes, so that such moisture, once inhaled, provides for relief of coughing. Such embodiments also reduce coughing exacerbated by dry air, for example.

In another humectant embodiment, a formulation contains a humectant such as sorbitol, glycerol, or other comparable compounds which facilitate structural and/or functional three dimensional disruption or disorientation of the viral envelope to permit the remaining ingredients of the formulation to penetrate the virus and affect rapid and irreversible destruction which kills the virus. The humectant should probably be used in the range of 20-80% w/v final concentration, preferably about 30% to 50% w/v final concentration, most preferably glycerol at about 40% w/v final concentration, or sorbitol at about 30% w/v final concentration. Activating the destruction of the envelope and the subsequent death of the envelope virus is achieved using a combination of inorganic monovalent anions, nonionic detergents and anionic detergents. These monovalent anions can include sodium bicarbonate, sodium thiocyanate, sodium fluoride and sodium chloride at about 0.5 to 5% w/v final concentration. The other ingredients include nonionic detergents such as Tween 20 at concentrations of about 0.1% to 3% v/v, ethanol up to 15% v/v and other antimicrobial agents can also be included, such as chlorhexidine or comparable basic substances at about 0.01 to 0.2% v/v. The compositions of the present invention also may include anionic surfactant detergent in a concentration of about 0.01 to about 3%, flavor and water added up to 100%. The compositions are embedded in the domes or enclosures that are inserted into the nostril. More details are at U.S. Pat. No. 5,213,803, the content of which is incorporated by reference.

In another embodiment, the filter can be coated with CBD or CBG and with camphor, menthol, eucalyptus, or other known natural cough suppressants that vaporize and permeate inhaled air as it enters the nasal passages. In another embodiment, the filter includes microencapsulated CDB/CBG, menthol, eucalyptus. In another embodiment, the filter has aromatherapy agents. In other embodiments, the filter is treated with other synthetic or natural compounds that are microencapsulated.

In some embodiments, the filter is made of multi-layer materials wherein any particular layer contributes to various methods of moisture retention and delivery for reducing or eliminating the effects of dry air, while other layers provide therapeutic compounds such as aromatherapy compounds, cough suppressants, nasal decongestants, or other medical or breathing treatments.

While a pair of elongate members 10 are shown, one embodiment provides a single member 10. In a disposable embodiment, a fabric pad of a breathable fabric layer attached to a base strip via adhesive and secured to the nose of an individual. The PPE is applied over one or both nostril openings to provide virus/pathogen air filtering, and/or volatile elements and compounds to the upper and lower respiratory system of an individual. The disposable PPE is a wearable, personal respiratory delivery system used to deliver filtered air, moisture, volatile medications and/or aromatherapy agents to the sinuses, pharynx, bronchial tree and lungs via inhalation. Preferably, a UV light source kills pathogen. The UV light is embedded in a UV safe shield to avoid skin cancer from UVA rays and UVB rays. UVB rays have more energy and are a more potent cause of at least some skin cancers, but both UVA and UVB rays can damage skin and cause skin cancer.

In another embodiment, the PPE provides for the slow, continuous administration of volatile elements and compounds to the upper and lower respiratory system of individuals. The nasal delivery device provides for a porous, highly permeable and breathable material and an adhesive that allows the device to be adhered to the lower portion of the nose covering the nostril openings (the external naris) and provides for easy removal. In one embodiment, the highly permeable material is comprised of hygroscopic fibers or humectant-coated fibers, to capture moisture from exhaled breath, and delivers the resulting moisture to the inhaled air. In another embodiment, volatile elements and compounds are incorporated into the device, coating the breathable material. The volatile compounds readily evaporate or sublimate at the temperatures and pressures found in the user's environment and are inhaled into the user's respiratory system.

In one embodiment, the PPE covers each nostril with a fabric pad affixed to a plastic film and secured to the nose by a skin-sensitive adhesive. In addition to the filtered/treated air, the PPE embodiment provides for an increase in the water vapor content of the air entering the nasal passages during normal inhalation. This application has the potential to ameliorate a condition known as nighttime cough, caused and exacerbated by low humidity of the air in the sleep environment. Two primary modes that provide for this increase in humidity are the use of humectant coatings on the breathable fabrics of the device or the incorporation of superabsorbent fibers within the breathable fabric structure.

Typical breathable fabrics for these applications are low density, open nonwoven structures such as spun-laced (hydroentangled) or needle-punched fabrics. These fabrics are coated with humectants (hygroscopic compounds) such as sugar alcohols (glycerol, sorbitol, maltitol, etc.), glycols (propylene glycol, butylene glycol, etc.) or even naturally occurring compounds such are Aloe Vera gel and honey. Such coated, breathable fabrics are either pre-moistened to supply moisture to the air inhaled through the device or supplied without pre-moistening. When supplied without pre-moistening, the humectant coating on the device captures and retains the moisture available in the high-humidity exhaled air that is then released during inhalation, increasing the humidity of the inhaled air. Breathable nonwoven materials are also available that contain superabsorbent and other hygroscopic fibers. Superabsorbents and superabsorbent fibers are water-absorbing polymers generally composed of sodium polyacrylates. These compounds are hygroscopic materials that can absorb and release water molecules to control humidity levels.

In another embodiment, a single nostril PPE provides a single fabric dome containing an air filter. Other functions can be added as well. For example, the single nostril PPE is utilized to deliver CBD/CBG material, a decongestant or other material within a single nostril. Alternatively, the single nostril delivery system can be used in pairs. The PPE has an adhesive layer on the opposite side from which the fabric dome 510 protrudes. The adhesive layer is a skin-sensitive material that provides for adhesion to the skin without causing irritation. The fabric dome provides for the absorption of moisture onto the particular fabric. As above, a humectant can be utilized to keep the fabric material moist, and the humectant can act as a filter that captures the corona virus. Water vapor is drawn into the humectant on the fabric surface. In another embodiment, sodium polyacrylate fiber is utilized to absorb moisture into the fabric. In various embodiments, humectants, sodium polyacrylate, and other such materials are utilized together to absorb moisture into the fabric dome, so that such moisture provides for relief of coughing. Such embodiments provide for alleviating coughing problems by alleviating dry air effects to minimize or eliminate dry air cough. As with the fabric pads described above, and in various embodiments, the fabric dome is a nonwoven manmade material, a woven manmade material, a nonwoven natural material, or a woven natural material. In another embodiment, the fibers of the fabric dome are coated with menthol, eucalyptus or other known natural cough suppressants. In other embodiments, the fabric dome 510 fibers include microencapsulated menthol, eucalyptus, or other known natural cough suppressants activated by the warmth of exhaled breath. In other embodiments, the fabric dome is treated with synthetic or natural compounds that are microencapsulated. In yet another embodiment, the fabric dome does not include sodium polyacrylate or other similar compounds that absorb and give up moisture. Such embodiments rely on naturally occurring materials and fibers to capture exhaled warm moisture, subsequently moisturizing and warming the air upon inhalation.

In some embodiments, the fabric dome is comprised of multi-layer materials, wherein any particular layer contributes to various moisture retention and/or other methods for reducing or eliminating the effects of dry air or providing cough suppressant medication, while other layers provide therapeutic compounds such as aromatherapy compounds, cough suppressants, nasal decongestants, or other medical or breathing treatments.

The single or dual nostril PPE is affixed to the nose by first centering the fabric dome over the nostril and then pressing the base strip firmly against the nasal septum and to the nasal tissue surrounding the nasal openings.

In one embodiment, the sensor can detect pathogens with nano sensors as detailed in U.S. Pat. No. 9,927,391 to the instant inventor Bao Tran, the content of which is incorporated by reference. Such sensors can detect pathogens with an upper metallic layer, a lower layer, and a nano sensor array positioned between the upper and lower layers to detect a presence of a gas, a chemical, or a biological object, wherein each sensor's electrical characteristic changes when encountering the gas, chemical or biological object. For example, In one aspect, a device includes an upper metallic layer, a lower layer, and a nano sensor array positioned between the upper and lower layers to detect a presence of a gas, a chemical, or a biological object, wherein each sensor's resistance changes when encountering the gas, chemical or biological object. In another aspect, a sensor device comprises an upper conductive layer, a non-metallic lower layer, and a switching matrix positioned between the upper and lower layers, said switching matrix having variable resistance based on one or more filaments in the switching matrix when voltage is applied to the upper and lower layers. In another aspect, a sensor device includes an upper conductive layer, a lower conductive layer, and a resistive, optical or magnetic matrix positioned between the upper and lower conductive layers. In yet another aspect, a sensor device includes an upper conductive layer, a lower conductive layer, and a memory resistive (memristive) matrix positioned between the upper and lower conductive layers. In a further aspect, a sensor device sensor device includes a first array of memory structures disposed in rows and columns and constructed over a substrate, each memory structure having a first signal electrode, a second signal electrode, and a resistive layer positioned between the first signal electrode and the second signal electrode. In another aspect, a sensor device includes a substrate; a first layer fabricated using semiconductor fabrication techniques; a second layer formed above the first layer, the second layer having one or more resistive-bonding areas and one or more resistive memory elements self-assembled to the second layer resistive bonding areas; and a third layer formed above the second layer, the third layer having one or more resistive-bonding areas and one or more resistive memory elements self-assembled to the third layer resistive bonding areas. In yet another aspect, a sensor device includes an array of memory structures disposed in rows and columns and constructed over a substrate, each memory structure comprising a first signal electrode, a second signal electrode, and a resistive layer coupled to the first signal electrode and the second signal electrode; a plurality of word lines connected to the first signal electrodes of a row of memory cells; and a plurality of bit lines connected to the second signal electrodes of a column of memory cells. The nano-elements can be formed last in the fabrication sequence in one embodiment. Conventional semiconductor structures are formed as is conventional, which for example includes semiconductor devices produced by photolithography or E-beam lithography. During the next to the last conventional step, gold electrodes are formed. Then a resist layer is formed over the last layer, and selective etching is performed to expose the gold electrodes. A solution containing the nano-elements are spin-coated on top, where the nano-elements self-assemble to form one or more devices such as resistors, capacitors, inductors, antennas, emitters and sensors, among others. Other coating techniques compatible with the present invention include hopper coating, curtain coating. The nano-elements bond to preselected spots on the gold electrodes and self-assemble to form a regular array of resistors, capacitors, inductors, acoustic emitters, acoustic sensors, light emitters, light sensors, among others. In one embodiment, the nano-elements do not need patterning. In another embodiment, patterning of the nano-elements is accomplished by any of the generally available photolithographic techniques utilized in semiconductor processing. However, depending on the particular material chosen, other techniques such as laser ablation or inkjet deposition or electrostatic deposition may also be utilized to pattern the nano-elements. In particular nanoimprint lithography can be used to pattern the nano-elements. In another embodiment, the substrate may be formed from silicon, gallium arsenide, indium phosphide, and silicon carbide to name a few. Active devices will be formed utilizing conventional semiconductor processing equipment. Other substrate materials can also be utilized, depending on the particular application in which the array will be used. For example various glasses, aluminum oxide and other inorganic dielectrics can be utilized. In addition, metals such as aluminum and tantalum that electrochemically form oxides, such as anodized aluminum or tantalum, can be utilized. Those applications utilizing non-semiconductor substrates, active devices can also be formed on these materials utilizing techniques such as amorphous silicon or polysilicon thin film transistor (TFT) processes or processes used to produce organic or polymer based active devices. Accordingly, the present system is not intended to be limited to those devices fabricated in silicon semiconductor materials, but will include those devices fabricated in one or more of the available semiconductor materials and technologies known in the art.

The process of creating the first layer of electrical conductors may consist of sputter deposition, electron beam evaporation, thermal evaporation, or chemical vapor deposition of either metals or alloys and will depend on the particular material chosen for the electrical conductors. Conductive materials such as polyaniline, polypyrrole, pentacene, thiophene compounds, or conductive inks, may utilize any of the techniques used to create thin organic films. For example, screen printing, spin coating, dip coating, spray coating, ink jet deposition and in some cases, as with PEDOT, thermal evaporation are techniques that may be used.

In another embodiment, the sensor(s) can collect vital signs such as temperature, heart rate, ECG, EEG, PPG, and bioimpedance, among others. For example, in one aspect, a system includes a cellular, WiFi, or and Bluetooth transceiver coupled to a processor; an accelerometer or a motion sensor coupled to the processor; and a sensor coupled to the processor to sense mood body vital sign, wherein text, image, sound, or video is rendered in response to a sensed mood or body vital sign; and a wearable device operating wirelessly with the processor, wherein the wearable device includes at least one sensor coupled to a back of the wearable device and wherein the wearable device recognizes and executes the speech command. In another aspect, a mobile system, comprising: a transceiver to communicate data via a personal area network (PAN); an accelerometer and a gyroscope; a processor coupled to the transceiver, the accelerometer and the gyroscope, the processor executing one or more applications to record user speech and to record data regarding movement detected by the accelerometer and the gyroscope; two or more sensors in communication with the processor to detect user vital sign data; and a health application executed by the processor to generate a health analysis using the vital sign data and the data regarding movement detected by the accelerometer and the gyroscope, wherein the transceiver communicates the analysis to another computer via the PAN. In yet another aspect, a system includes a processor; a cellular, WiFi, or Bluetooth transceiver coupled to the processor; an accelerometer or a motion sensor coupled to the processor; and a sensor coupled to the processor to sense mood, wherein text, image, sound, or video is rendered in response to the sensed mood. In another aspect, a system includes an accelerometer to detect movement or fitness; a sensor coupled to a wrist, hand or finger to detect blood-oxygen levels or heart rate or pulse rate and mounted on a wristwatch wearable device and a voice communication device having a wireless transceiver adapted to receive blood-oxygen level or heart rate or pulse rate from the sensor over a wireless personal area network (PAN). In yet another aspect, a system includes a cellular telephone having a vital sign sensor thereon to detect heart rate, pulse rate or blood-oxygen levels; and a wristwatch wearable device in wireless communication with the cellular telephone, including: a sensor coupled to a wrist, hand or finger to detect blood-oxygen levels, heart rate or pulse rate; a wireless transceiver adapted to communicate with the cellular telephone over a wireless personal area network (PAN); and a processor coupled to the sensor and the transceiver to send pulse rate to the cellular telephone. In a further aspect, a health care monitoring system for a person includes one or more wireless nodes forming a wireless network to communicate data over the wireless network to detect a health problem. Implementations can include watches that capture fitness data (activity, heart rate, blood pressure, walking rate, dietary or calorie consumption, among others) and sending the data to a hospital database where medical and fitness data is used to treat the patient. Other implementations include collecting data from different devices with different communication protocols such as blood pressure measurement devices, scales, glucose meters, among others, and upload the data to a computer which converts the data into an intermediate format that is compatible with different protocols for interoperability purposes. In another aspect, a heart monitoring system for a person includes one or more wireless nodes forming a wireless network; a wearable sensor having a wireless transceiver adapted to communicate with the one or more wireless nodes; and a software module receiving data from the wireless nodes to detect changes in patient vital signs. In another aspect, a monitoring system includes one or more wireless nodes forming a wireless network; a wearable blood pressure sensor having a wireless transceiver adapted to communicate with the one or more wireless nodes; and a software module receiving data from the wireless nodes to detect deteriorations in patient vital signs. In another aspect, a health care monitoring system for a person includes one or more wireless nodes forming a wireless mesh network; a wearable appliance having a sound transducer coupled to the wireless transceiver; and a bioelectric impedance (BI) sensor coupled to the wireless mesh network to communicate BI data over the wireless mesh network. In another aspect, a heart monitoring system for a person includes one or more wireless nodes forming a wireless mesh network and a wearable appliance having a sound transducer coupled to the wireless transceiver; and a heart disease recognizer coupled to the sound transducer to determine cardiovascular health and to transmit heart sound over the wireless mesh network to a remote listener if the recognizer identifies a cardiovascular problem. The heart sound being transmitted may be compressed to save transmission bandwidth. In yet another aspect, a monitoring system for a person includes one or more wireless nodes; and a wristwatch having a wireless transceiver adapted to communicate with the one or more wireless nodes; and an accelerometer to detect a dangerous condition and to generate a warning when the dangerous condition is detected. In yet another aspect, a monitoring system for a person includes one or more wireless nodes forming a wireless mesh network; and a wearable appliance having a wireless transceiver adapted to communicate with the one or more wireless nodes; and a heartbeat detector coupled to the wireless transceiver. The system may also include an accelerometer to detect a dangerous condition such as a falling condition and to generate a warning when the dangerous condition is detected. In yet another aspect, a monitoring system for a person includes one or more wireless nodes forming a wireless network; and a wearable device including: a processor; a transceiver coupled to the processor to communicate with the one or more wireless nodes; a wearable sensor on a patch or bandage secured to the person's skin and coupled to the processor; an accelerometer coupled to the processor; and a thumb sensor coupled to the processor. In another aspect, a health monitoring system for a person includes a mobile telephone case including a cellular transceiver to provide wireless data and voice communication; a sensor including one or more electrodes mounted on the mobile telephone case to contact the person's skin and capture bio-electrical signals therefrom; an amplifier coupled to the electrodes; a processor coupled to the amplifier; and a screen coupled to the processor to display medical data such as images of the bio-electrical signals. Implementations of the above aspect may include one or more of the following. The wristwatch determines position based on triangulation. The wristwatch determines position based on RF signal strength and RF signal angle. A switch detects a confirmatory signal from the person. The confirmatory signal includes a head movement, a hand movement, or a mouth movement. The confirmatory signal includes the person's voice. A processor in the system executes computer readable code to transmit a help request to a remote computer. The code can encrypt or scramble data for privacy. The processor can execute voice over IP (VOIP) code to allow a user and a remote person to audibly communicate with each other. The voice communication system can include Zigbee VOIP or Bluetooth VOIP or 802.XX VOIP. The remote person can be a doctor, a nurse, a medical assistant, or a caregiver. The system includes code to store and analyze patient information. The patient information includes medicine taking habits, eating and drinking habits, sleeping habits, or excise habits. A patient interface is provided on a user computer for accessing information and the patient interface includes in one implementation a touch screen; voice-activated text reading; and one touch telephone dialing. The processor can execute code to store and analyze information relating to the person's ambulation. A global positioning system (GPS) receiver can be used to detect movement and where the person falls. The system can include code to map the person's location onto an area for viewing. The system can include one or more cameras positioned to capture three dimensional (3D) video of the patient; and a server coupled to the one or more cameras, the server executing code to detect a dangerous condition for the patient based on the 3D video and allow a remote third party to view images of the patient when the dangerous condition is detected. In another aspect, a monitoring system for a person includes one or more wireless bases; and a cellular telephone having a wireless transceiver adapted to communicate with the one or more wireless bases; and an accelerometer to detect a dangerous condition and to generate a warning when the dangerous condition is detected. In one aspect, systems and methods include one or more entities including a sensor configured to provide data in at least a first information standard from a first manufacturer and a second information standard from a second manufacturer; and an electronic health record database configured to: capture information from the one or more entities, normalize the captured information from first and second manufacturers in a common format, and add metadata for the captured information. In another aspect, an interoperable health-care system includes a network; one or more medical data collection appliances coupled to the network, each appliance transmitting data conforming to an interoperable format; and a computer coupled to the network to store data for each individual in accordance with the interoperable format. The user can take his/her weight, blood pressure, and cholesterol measurement daily, and the data is sent from a health base station to a monitoring service at his doctor's office. Periodically, the user gets an automated health summary generated by a service at his doctor's office as well as information to help him maintain a healthy lifestyle. The health information can be stored in an external HIPAA compliant health storage database so that the user and his doctor can access his health information over the web. The system extends health care system into the home and can record personal health data on a systematic periodic basis. Appointments can be automatically scheduled with providers. Long-term data for medical baseline can be collected. The system can also provide predictive alerts for high-risk conditions. The system can perform initial triage utilizing biosensors, images, e-mail/chat/video.

By enabling a network of readily connected health and medical devices, people with Covid or infectious disease or other chronic diseases will be able to share vital sign information such as blood pressure and glucose level with their doctors. Adult children will be able to remotely watch over their aging parents and proactively help them manage safely in their own homes. Diet and fitness conscious individuals will also be able to seamlessly share their weight and exercise data with fitness consultants through the Internet. The above system forms an interoperable health-care system with a network; a first medical appliance to capture a first vital information and coupled to the network, the first medical appliance transmitting the first vital information conforming to an interoperable format; and a second medical appliance to capture a second vital information and coupled to the network, the second medical appliance converting the first vital information in accordance with the interoperable format and processing the first and second vital information, the second medical appliance providing an output conforming to the interoperable format. The appliances can communicate data conforming to the interoperable format over one of: cellular protocol, ZigBee protocol, Bluetooth protocol, WiFi protocol, WiMAX protocol, USB protocol, ultrawideband protocol. The appliances can communicate over two or more protocols. The first medical appliance can transmit the first vital information over a first protocol (such as Bluetooth protocol) to a computer, wherein the computer transmits the first vital information to the second medical appliance over a second protocol (such as ZigBee prototocol). The computer can then transmit to a hospital or physician office using broadband such as WiMAX protocol or cellular protocol. The computer can perform the interoperable format conversion for the appliances or devices, or alternatively each appliance or device can perform the format conversion. Regardless of which device performs the protocol conversion and format conversion, the user does not need to know about the underlying format or protocol in order to use the appliances. The user only needs to plug an appliance into the network, the data transfer is done automatically so that the electronic “plumbing” is not apparent to the user. In this way, the user is shielded from the complexity supporting interoperability. In another aspect, a monitoring system for a person includes one or more wireless nodes and a stroke sensor coupled to the person and the wireless nodes to determine a medical problem, for example a stroke attack. The stroke monitoring system is interoperable with emergency vehicle and/or hospital systems and provides information to quickly treat stroke once the patient reaches the treatment center.

In one aspect, a monitoring system for a person includes one or more wireless nodes and an electromyography (EMG) sensor coupled to the person and the wireless nodes to determine a medical issue such as a stroke attack. In another aspect, a health care monitoring system for a person includes one or more wireless nodes forming a wireless mesh network; a wearable appliance having a sound transducer coupled to the wireless transceiver; and a bioelectric impedance (BI) sensor coupled to the wireless mesh network to communicate BI data over the wireless mesh network. In a further aspect, a heart monitoring system for a person includes one or more wireless nodes forming a wireless mesh network and a wearable appliance having a sound transducer coupled to the wireless transceiver; and a heart disease recognizer coupled to the sound transducer to determine cardiovascular health and to transmit heart sound over the wireless mesh network to a remote listener if the recognizer identifies a cardiovascular problem. The heart sound being transmitted may be compressed to save transmission bandwidth. In yet another aspect, a monitoring system for a person includes one or more wireless nodes; and a wristwatch having a wireless transceiver adapted to communicate with the one or more wireless nodes; and an accelerometer to detect a dangerous condition and to generate a warning when the dangerous condition is detected. In yet another aspect, a monitoring system for a person includes one or more wireless nodes forming a wireless mesh network; and a wearable appliance having a wireless transceiver adapted to communicate with the one or more wireless nodes; and a heartbeat detector coupled to the wireless transceiver. The system may also include an accelerometer to detect a dangerous condition such as a falling condition and to generate a warning when the dangerous condition is detected. Implementations of the above aspect may include one or more of the following. The wristwatch determines position based on triangulation. The wristwatch determines position based on RF signal strength and RF signal angle. A switch detects a confirmatory signal from the person. The confirmatory signal includes a head movement, a hand movement, or a mouth movement. The confirmatory signal includes the person's voice. A processor in the system executes computer readable code to transmit a help request to a remote computer. The code can encrypt or scramble data for privacy. The processor can execute voice over IP (VOIP) code to allow a user and a remote person to audibly communicate with each other. The voice communication system can include Zigbee VOIP or Bluetooth VOIP or 802.XX VOIP. The remote person can be a doctor, a nurse, a medical assistant, or a caregiver. The system includes code to store and analyze patient information. The patient information includes medicine taking habits, eating and drinking habits, sleeping habits, or excise habits. A patient interface is provided on a user computer for accessing information and the patient interface includes in one implementation a touch screen; voice-activated text reading; and one touch telephone dialing. The processor can execute code to store and analyze information relating to the person's ambulation. A global positioning system (GPS) receiver can be used to detect movement and where the person falls. The system can include code to map the person's location onto an area for viewing. The system can include one or more cameras positioned to capture three dimensional (3D) video of the patient; and a server coupled to the one or more cameras, the server executing code to detect a dangerous condition for the patient based on the 3D video and allow a remote third party to view images of the patient when the dangerous condition is detected.

One embodiment includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies. The bioimpedance sensor can detect body fluid build up, and can be used to estimate glucose levels, among others. More details on the use of bioimpedance are disclosed in Patent Application Ser. 62/930,305 by Bao Tran, the content of which are incorporated by reference.

In one implementation, BIA measurements can be made using an array of bipolar or tetrapolar electrodes that deliver a constant alternating current at 50 KHz frequency. Whole body measurements can be done using standard right-sided. The ability of any biological tissue to resist a constant electric current depends on the relative proportions of water and electrolytes it contains, and is called resistivity (in Ohms/cm 3). The measuring of bioimpedance to assess congestive heart failure employs the different bio-electric properties of blood and lung tissue to permit separate assessment of: (a) systemic venous congestion via a low frequency or direct current resistance measurement of the current path through the right ventricle, right atrium, superior vena cava, and subclavian vein, or by computing the real component of impedance at a high frequency, and (b) pulmonary congestion via a high frequency measurement of capacitive impedance of the lung. The resistance is impedance measured using direct current or alternating current (AC) which can flow through capacitors.

In one embodiment, a belt is worn by the patient with a plurality of BI probes positioned around the belt perimeter. The output of the tetrapolar probes is processed using a second-order Newton-Raphson method to estimate the left and right-lung resistivity values in the thoracic geometry. The locations of the electrodes are marked. During the measurements procedure, the belt is worn around the patient's thorax while sitting, and the reference electrode is attached to his waist. The data is collected during tidal respiration to minimize lung resistivity changes due to breathing, and lasts approximately one minute. The process is repeated periodically and the impedance trend is analyzed to detect CHF. Upon detection, the system provides vital parameters to a call center and the call center can refer to a physician for consultation or can call 911 for assistance.

In one embodiment, an array of noninvasive thoracic electrical bioimpedance monitoring probes can be used alone or in conjunction with other techniques such as impedance cardiography (ICG) for early comprehensive cardiovascular assessment and trending of acute trauma victims. This embodiment provides early, continuous cardiovascular assessment to help identify patients whose injuries were so severe that they were not likely to survive. This included severe blood and/or fluid volume deficits induced by trauma, which did not respond readily to expeditious volume resuscitation and vasopressor therapy. One exemplary system monitors cardiorespiratory variables that served as statistically significant measures of treatment outcomes: Qt, BP, pulse oximetry, and transcutaneous Po2 (Ptco2). A high Qt may not be sustainable in the presence of hypovolemia, acute anemia, pre-existing impaired cardiac function, acute myocardial injury, or coronary ischemia. Thus a fall in Ptco2 could also be interpreted as too high a metabolic demand for a patient's cardiovascular reserve. Too high a metabolic demand may compromise other critical organs. Acute lung injury from hypotension, blunt trauma, and massive fluid resuscitation can drastically reduce respiratory reserve.

One embodiment that measures thoracic impedance (a resistive or reactive impedance associated with at least a portion of a thorax of a living organism). The thoracic impedance signal is influenced by the patient's thoracic intravascular fluid tension, heart beat, and breathing (also referred to as “respiration” or “ventilation”). A “de” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive). A low intravascular fluid tension in the thorax (“thoracic hypotension”) may result from changes in posture. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

The accelerometer can be used to provide reproducible measurements. Body activity will increase cardiac output and also change the amount of blood in the systemic venous system or lungs. Measurements of congestion may be most reproducible when body activity is at a minimum and the patient is at rest. The use of an accelerometer allows one to sense both body position and body activity. Comparative measurements over time may best be taken under reproducible conditions of body position and activity. Ideally, measurements for the upright position should be compared as among themselves. Likewise measurements in the supine, prone, left lateral decubitus and right lateral decubitus should be compared as among themselves. Other variables can be used to permit reproducible measurements, i.e. variations of the cardiac cycle and variations in the respiratory cycle. The ventricles are at their most compliant during diastole. The end of the diastolic period is marked by the QRS on the electrocardiographic means (EKG) for monitoring the cardiac cycle. The second variable is respiratory variation in impedance, which is used to monitor respiratory rate and volume. As the lungs fill with air during inspiration, impedance increases, and during expiration, impedance decreases. Impedance can be measured during expiration to minimize the effect of breathing on central systemic venous volume. While respiration and CHF both cause variations in impedance, the rates and magnitudes of the impedance variation are different enough to separate out the respiratory variations which have a frequency of about 8 to 60 cycles per minute and congestion changes which take at least several minutes to hours or even days to occur. Also, the magnitude of impedance change is likely to be much greater for congestive changes than for normal respiratory variation. Thus, the system can detect congestive heart failure (CHF) in early stages and alert a patient to prevent disabling and even lethal episodes of CHF. Early treatment can avert progression of the disorder to a dangerous stage.

In an embodiment to monitor wounds such as diabetic related wounds, the conductivity of a region of the patient with a wound or is susceptible to wound formation is monitored by the system. The system determines healing wounds if the impedance and reactance of the wound region increases as the skin region becomes dry. The system detects infected, open, interrupted healing, or draining wounds through lower regional electric impedances. In yet another embodiment, the bioimpedance sensor can be used to determine body fat. In one embodiment, the BI system determines Total Body Water (TBW) which is an estimate of total hydration level, including intracellular and extracellular water; Intracellular Water (ICW) which is an estimate of the water in active tissue and as a percent of a normal range (near 60% of TBW); Extracellular Water (ECW) which is water in tissues and plasma and as a percent of a normal range (near 40% of TBW); Body Cell Mass (BCM) which is an estimate of total pounds/kg of all active cells; Extracellular Tissue (ECT)/Extracellular Mass (ECM) which is an estimate of the mass of all other non-muscle inactive tissues including ligaments, bone and ECW; Fat Free Mass (FFM)/Lean Body Mass (LBM) which is an estimate of the entire mass that is not fat. It should be available in pounds/kg and may be presented as a percent with a normal range; Fat Mass (FM) which is an estimate of pounds/kg of body fat and percentage body fat; and Phase Angle (PA) which is associated with both nutrition and physical fitness.

Additional sensors such as thermocouples or thermisters and/or heat flux sensors can also be provided to provide measured values useful in analysis. In general, skin surface temperature will change with changes in blood flow in the vicinity of the skin surface of an organism. Such changes in blood flow can occur for a number of reasons, including thermal regulation, conservation of blood volume, and hormonal changes. In one implementation, skin surface measurements of temperature or heat flux are made in conjunction with hydration monitoring so that such changes in blood flow can be detected and appropriately treated.

In one embodiment, the patch includes a sound transducer such as a microphone or a piezoelectric transducer to pick up sound produced by bones or joints during movement. If bone surfaces are rough and poorly lubricated, as in an arthritic knee, they will move unevenly against each other, producing a high-frequency, scratching sound. The high-frequency sound from joints is picked up by wide-band acoustic sensor(s) or microphone(s) on a patient's body such as the knee. As the patient flexes and extends their knee, the sensors measure the sound frequency emitted by the knee and correlate the sound to monitor osteoarthritis, for example.

In another embodiment, the patch includes a Galvanic Skin Response (GSR) sensor. In this sensor, a small current is passed through one of the electrodes into the user's body such as the fingers and the CPU calculates how long it takes for a capacitor to fill up. The length of time the capacitor takes to fill up allows us to calculate the skin resistance: a short time means low resistance while a long time means high resistance. The GSR reflects sweat gland activity and changes in the sympathetic nervous system and measurement variables. Measured from the palm or fingertips, there are changes in the relative conductance of a small electrical current between the electrodes. The activity of the sweat glands in response to sympathetic nervous stimulation (Increased sympathetic activation) results in an increase in the level of conductance. Fear, anger, startle response, orienting response and sexual feelings are all among the emotions which may produce similar GSR responses.

In yet another embodiment, measurement of lung function such as peak expiratory flow readings is done though a sensor such as Wright's peak flow meter. In another embodiment, a respiratory estimator is provided that avoids the inconvenience of having the patient breathing through the flow sensor. In the respiratory estimator embodiment, heart period data from EKG/ECG is used to extract respiratory detection features. The heart period data is transformed into time-frequency distribution by applying a time-frequency transformation such as short-term Fourier transformation (STFT). Other possible methods are, for example, complex demodulation and wavelet transformation. Next, one or more respiratory detection features may be determined by setting up amplitude modulation of time-frequency plane, among others. The respiratory recognizer first generates a math model that correlates the respiratory detection features with the actual flow readings. The math model can be adaptive based on pre-determined data and on the combination of different features to provide a single estimate of the respiration. The estimator can be based on different mathematical functions, such as a curve fitting approach with linear or polynomical equations, and other types of neural network implementations, non-linear models, fuzzy systems, time series models, and other types of multivariate models capable of transferring and combining the information from several inputs into one estimate. Once the math model has been generated, the respirator estimator provides a real-time flow estimate by receiving EKG/ECG information and applying the information to the math model to compute the respiratory rate. Next, the computation of ventilation uses information on the tidal volume. An estimate of the tidal volume may be derived by utilizing different forms of information on the basis of the heart period signal. For example, the functional organization of the respiratory system has an impact in both respiratory period and tidal volume. Therefore, given the known relationships between the respiratory period and tidal volume during and transitions to different states, the information inherent in the heart period derived respiratory frequency may be used in providing values of tidal volume. In specific, the tidal volume contains inherent dynamics which may be, after modeling, applied to capture more closely the behavioral dynamics of the tidal volume. Moreover, it appears that the heart period signal, itself, is closely associated with tidal volume and may be therefore used to increase the reliability of deriving information on tidal volume. The accuracy of the tidal volume estimation may be further enhanced by using information on the subjects vital capacity (i.e., the maximal quantity of air that can be contained in the lungs during one breath). The information on vital capacity, as based on physiological measurement or on estimates derived from body measures such as height and weight, may be helpful in estimating tidal volume, since it is likely to reduce the effects of individual differences on the estimated tidal volume. Using information on the vital capacity, the mathematical model may first give values on the percentage of lung capacity in use, which may be then transformed to liters per breath. The optimizing of tidal volume estimation can based on, for example, least squares or other type of fit between the features and actual tidal volume. The minute ventilation may be derived by multiplying respiratory rate (breaths/min) with tidal volume (liters/breath).

In another embodiment, inductive plethysmography can be used to measure a cross-sectional area of the body by determining the self-inductance of a flexible conductor closely encircling the area to be measured. Since the inductance of a substantially planar conductive loop is well known to vary as, inter alia, the cross-sectional area of the loop, a inductance measurement may be converted into a plethysmographic area determination. Varying loop inductance may be measured by techniques known in the art, such as, e.g., by connecting the loop as the inductance in a variable frequency LC oscillator, the frequency of the oscillator then varying with the cross-sectional area of the loop inductance varies. Oscillator frequency is converted into a digital value, which is then further processed to yield the physiological parameters of interest. Specifically, a flexible conductor measuring a cross-sectional area of the body is closely looped around the area of the body so that the inductance, and the changes in inductance, being measured results from magnetic flux through the cross-sectional area being measured. The inductance thus depends directly on the cross-sectional area being measured, and not indirectly on an area which changes as a result of the factors changing the measured cross-sectional area. Various physiological parameters of medical and research interest may be extracted from repetitive measurements of the areas of various cross-sections of the body. For example, pulmonary function parameters, such as respiration volumes and rates and apneas and their types, may be determined from measurements of, at least, a chest transverse cross-sectional area and also an abdominal transverse cross-sectional area. Cardiac parameters, such central venous pressure, left and right ventricular volumes waveforms, and aortic and carotid artery pressure waveforms, may be extracted from repetitive measurements of transverse cross-sectional areas of the neck and of the chest passing through the heart. Timing measurements can be obtained from concurrent ECG measurements, and less preferably from the carotid pulse signal present in the neck. From the cardiac-related signals, indications of ischemia may be obtained independently of any ECG changes. Ventricular wall ischemia is known to result in paradoxical wall motion during ventricular contraction (the ischemic segment paradoxically “balloons” outward instead of normally contracting inward). Such paradoxical wall motion, and thus indications of cardiac ischemia, may be extracted from chest transverse cross-section area measurements. Left or right ventricular ischemia may be distinguished where paradoxical motion is seen predominantly in left or right ventricular waveforms, respectively. For another example, observations of the onset of contraction in the left and right ventricles separately may be of use in providing feedback to bi-ventricular cardiac pacing devices. For a further example, pulse oximetry determines hemoglobin saturation by measuring the changing infrared optical properties of a finger. This signal may be disambiguated and combined with pulmonary data to yield improved information concerning lung function.

In one embodiment, the CPU produces the estimate of heartbeat rate by first averaging a plurality of measurements, then adjusting the particular one of the measurements that differs most from the average to be equal to that average, and finally computing an adjusted average based on the adjusted set of measurements. The process may repeat the foregoing operations a number of times so that the estimate of heartbeat rate is substantially unaffected by the occurrence of heartbeat artifacts.

In one EKG or ECG detector, the heartbeat detection circuitry includes a differential amplifier for amplifying the signal transmitted from the EKG/ECG electrodes and for converting it into single-ended form, and a bandpass filter and a 60 Hz notch filter for removing background noise. The CPU measures the time durations between the successive pulses and estimates the heartbeat rate. The time durations between the successive pulses of the pulse sequence signal provides an estimate of heartbeat rate. Each time duration measurement is first converted to a corresponding rate, preferably expressed in beats per minute (bpm), and then stored in a file, taking the place of the earliest measurement previously stored. After a new measurement is entered into the file, the stored measurements are averaged, to produce an average rate measurement. The CPU optionally determines which of the stored measurements differs most from the average, and replaces that measurement with the average.

Upon initiation, the CPU increments a period timer used in measuring the time duration between successive pulses. This timer is incremented in steps of about two milliseconds in one embodiment. It is then determined whether or not a pulse has occurred during the previous two milliseconds. If it has not, the CPU returns to the initial step of incrementing the period timer. If a heartbeat has occurred, on the other hand, the CPU converts the time duration measurement currently stored in the period timer to a corresponding heartbeat rate, preferably expressed in bpm. After the heartbeat rate measurement is computed, the CPU determines whether or not the computed rate is intermediate prescribed thresholds of 20 bpm and 240 bpm. If it is not, it is assumed that the detected pulse was not in fact a heartbeat and the period timer is cleared.

In an optical heartbeat detector embodiment, an optical transducer is positioned on a finger, wrist, or ear lobe. The ear, wrist or finger pulse oximeter waveform is then analyzed to extract the beat-to-beat amplitude, area, and width (half height) measurements. The oximeter waveform is used to generate heartbeat rate in this embodiment. In one implementation, a reflective sensor such as the Honeywell HLC1395 can be used. The device emits lights from a window in the infrared spectrum and receives reflected light in a second window. When the heart beats, blood flow increases temporarily and more red blood cells flow through the windows, which increases the light reflected back to the detector. The light can be reflected, refracted, scattered, and absorbed by one or more detectors. Suitable noise reduction is done, and the resulting optical waveform is captured by the CPU.

In another optical embodiment, blood pressure is estimated from the optical reading using a mathematical model such as a linear correlation with a known blood pressure reading. In this embodiment, the pulse oximeter readings are compared to the blood-pressure readings from a known working blood pressure measurement device during calibration. Using these measurements the linear equation is developed relating oximeter output waveform such as width to blood-pressure (systolic, mean and pulse pressure). In one embodiment, a transform (such as a Fourier analysis or a Wavelet transform) of the oximeter output can be used to generate a model to relate the oximeter output waveform to the blood pressure. Other non-linear math model or relationship can be determined to relate the oximeter waveform to the blood pressure.

In one implementation, the pulse oximeter probe and a blood pressure cuff are placed on the corresponding contralateral limb to the oscillometric (Dinamap 8100; Critikon, Inc, Tampa, Fla., USA) cuff site. The pulse oximeter captures data on plethysmographic waveform, heart rate, and oxygen saturation. Simultaneous blood pressure measurements were obtained from the oscillometric device, and the pulse oximeter. Systolic, diastolic, and mean blood pressures are recorded from the oscillometric device. This information is used derive calibration parameters relating the pulse oximeter output to the expected blood pressure. During real time operation, the calibration parameters are applied to the oximeter output to predict blood pressure in a continuous or in a periodic fashion. In yet another embodiment, the device includes an accelerometer or alternative motion-detecting device to determine when the patient' hand is at rest, thereby reducing motion-related artifacts introduced to the measurement during calibration and/or operation. The accelerometer can also function as a falls detection device.

In an ultrasonic embodiment, a piezo film sensor element is placed on the wristwatch band. The sensor can be the SDT1-028K made by Measurement Specialties, Inc. The sensor should have features such as: (a) it is sensitive to low level mechanical movements, (b) it has an electrostatic shield located on both sides of the element (to minimize 50/60 Hz AC line interference), (c) it is responsive to low frequency movements in the 0.7-12 Hz range of interest. A filter/amplifier circuit has a three-pole low pass filter with a lower (−3 dB) cutoff frequency at about 12-13 Hz. The low-pass filter prevents unwanted 50/60 Hz AC line interference from entering the sensor. However, the piezo film element has a wide band frequency response so the filter also attenuates any extraneous sound waves or vibrations that get into the piezo element. The DC gain is about +30 dB.

In one embodiment, once the heart sound signal has been digitized and captured into the memory, the digitized heart sound signal is parameterized into acoustic features by a feature extractor. The output of the feature extractor is delivered to a sound recognizer. The feature extractor can include the short time energy, the zero crossing rates, the level crossing rates, the filter-bank spectrum, the linear predictive coding (LPC), and the fractal method of analysis. In addition, vector quantization may be utilized in combination with any representation techniques. Further, one skilled in the art may use an auditory signal-processing model in place of the spectral models to enhance the system's robustness to noise and reverberation.

In one embodiment of the feature extractor, the digitized heart sound signal series s(n) is put through a low-order filter, typically a first-order finite impulse response filter, to spectrally flatten the signal and to make the signal less susceptible to finite precision effects encountered later in the signal processing. The signal is pre-emphasized preferably using a fixed pre-emphasis network, or preemphasizer. The signal can also be passed through a slowly adaptive pre-emphasizer. The preemphasized heart sound signal is next presented to a frame blocker to be blocked into frames of N samples with adjacent frames being separated by M samples. In one implementation, frame 1 contains the first 400 samples. The frame 2 also contains 400 samples, but begins at the 300th sample and continues until the 700th sample. Because the adjacent frames overlap, the resulting LPC spectral analysis will be correlated from frame to frame. Each frame is windowed to minimize signal discontinuities at the beginning and end of each frame. The windower tapers the signal to zero at the beginning and end of each frame. Preferably, the window used for the autocorrelation method of LPC is the Hamming window. A noise canceller operates in conjunction with the autocorrelator to minimize noise. Noise in the heart sound pattern is estimated during quiet periods, and the temporally stationary noise sources are damped by means of spectral subtraction, where the autocorrelation of a clean heart sound signal is obtained by subtracting the autocorrelation of noise from that of corrupted heart sound. In the noise cancellation unit, if the energy of the current frame exceeds a reference threshold level, the heart is generating sound and the autocorrelation of coefficients representing noise is not updated. However, if the energy of the current frame is below the reference threshold level, the effect of noise on the correlation coefficients is subtracted off in the spectral domain. The result is half-wave rectified with proper threshold setting and then converted to the desired autocorrelation coefficients. The output of the autocorrelator and the noise canceller are presented to one or more parameterization units, including an LPC parameter unit, an FFT parameter unit, an auditory model parameter unit, a fractal parameter unit, or a wavelet parameter unit, among others. The LPC parameter is then converted into cepstral coefficients. The cepstral coefficients are the coefficients of the Fourier transform representation of the log magnitude spectrum. A filter bank spectral analysis, which uses the short-time Fourier transformation (STFT) may also be used alone or in conjunction with other parameter blocks. FFT is well known in the art of digital signal processing. Such a transform converts a time domain signal, measured as amplitude over time, into a frequency domain spectrum, which expresses the frequency content of the time domain signal as a number of different frequency bands. The FFT thus produces a vector of values corresponding to the energy amplitude in each of the frequency bands. The FFT converts the energy amplitude values into a logarithmic value which reduces subsequent computation since the logarithmic values are more simple to perform calculations on than the longer linear energy amplitude values produced by the FFT, while representing the same dynamic range. Ways for improving logarithmic conversions are well known in the art, one of the simplest being use of a look-up table. In addition, the FFT modifies its output to simplify computations based on the amplitude of a given frame. This modification is made by deriving an average value of the logarithms of the amplitudes for all bands. This average value is then subtracted from each of a predetermined group of logarithms, representative of a predetermined group of frequencies. The predetermined group consists of the logarithmic values, representing each of the frequency bands. Thus, utterances are converted from acoustic data to a sequence of vectors of k dimensions, each sequence of vectors identified as an acoustic frame, each frame represents a portion of the utterance. Alternatively, auditory modeling parameter unit can be used alone or in conjunction with others to improve the parameterization of heart sound signals in noisy and reverberant environments. In this approach, the filtering section may be represented by a plurality of filters equally spaced on a log-frequency scale from 0 Hz to about 3000 Hz and having a prescribed response corresponding to the cochlea. The nerve fiber firing mechanism is simulated by a multilevel crossing detector at the output of each cochlear filter. The ensemble of the multilevel crossing intervals corresponds to the firing activity at the auditory nerve fiber-array. The interval between each successive pair of same direction, either positive or negative going, crossings of each predetermined sound intensity level is determined and a count of the inverse of these interspike intervals of the multilevel detectors for each spectral portion is stored as a function of frequency. The resulting histogram of the ensemble of inverse interspike intervals forms a spectral pattern that is representative of the spectral distribution of the auditory neural response to the input sound and is relatively insensitive to noise. The use of a plurality of logarithmically related sound intensity levels accounts for the intensity of the input signal in a particular frequency range. Thus, a signal of a particular frequency having high intensity peaks results in a much larger count for that frequency than a low intensity signal of the same frequency. The multiple level histograms of the type described herein readily indicate the intensity levels of the nerve firing spectral distribution and cancel noise effects in the individual intensity level histograms. Alternatively, the fractal parameter block can further be used alone or in conjunction with others to represent spectral information. Fractals have the property of self similarity as the spatial scale is changed over many orders of magnitude. A fractal function includes both the basic form inherent in a shape and the statistical or random properties of the replacement of that shape in space. As is known in the art, a fractal generator employs mathematical operations known as local affine transformations. These transformations are employed in the process of encoding digital data representing spectral data. The encoded output constitutes a “fractal transform” of the spectral data and consists of coefficients of the affine transformations. Different fractal transforms correspond to different images or sounds.

Alternatively, a wavelet parameterization block can be used alone or in conjunction with others to generate the parameters. Like the FFT, the discrete wavelet transform (DWT) can be viewed as a rotation in function space, from the input space, or time domain, to a different domain. The DWT consists of applying a wavelet coefficient matrix hierarchically, first to the full data vector of length N, then to a smooth vector of length N/2, then to the smooth-smooth vector of length N/4, and so on. Most of the usefulness of wavelets rests on the fact that wavelet transforms can usefully be severely truncated, or turned into sparse expansions. In the DWT parameterization block, the wavelet transform of the heart sound signal is performed. The wavelet coefficients are allocated in a non-uniform, optimized manner. In general, large wavelet coefficients are quantized accurately, while small coefficients are quantized coarsely or even truncated completely to achieve the parameterization. Due to the sensitivity of the low-order cepstral coefficients to the overall spectral slope and the sensitivity of the high-order cepstral coefficients to noise variations, the parameters generated may be weighted by a parameter weighing block, which is a tapered window, so as to minimize these sensitivities. Next, a temporal derivator measures the dynamic changes in the spectra. Power features are also generated to enable the system to distinguish heart sound from silence.

After the feature extraction has been performed, the heart sound parameters are next assembled into a multidimensional vector and a large collection of such feature signal vectors can be used to generate a much smaller set of vector quantized (VQ) feature signals by a vector quantizer that cover the range of the larger collection. In addition to reducing the storage space, the VQ representation simplifies the computation for determining the similarity of spectral analysis vectors and reduces the similarity computation to a look-up table of similarities between pairs of codebook vectors. To reduce the quantization error and to increase the dynamic range and the precision of the vector quantizer, the preferred embodiment partitions the feature parameters into separate codebooks, preferably three. In the preferred embodiment, the first, second and third codebooks correspond to the cepstral coefficients, the differenced cepstral coefficients, and the differenced power coefficients.

With conventional vector quantization, an input vector is represented by the codeword closest to the input vector in terms of distortion. In conventional set theory, an object either belongs to or does not belong to a set. This contrasts with fuzzy sets where the membership of an object to a set is not so clearly defined so that the object can be a part member of a set. Data are assigned to fuzzy sets based upon the degree of membership therein, which ranges from 0 (no membership) to 1.0 (full membership). A fuzzy set theory uses membership functions to determine the fuzzy set or sets to which a data value belongs and its degree of membership therein.

To handle the variance of heart sound patterns of individuals over time and to perform speaker adaptation in an automatic, self-organizing manner, an adaptive clustering technique called hierarchical spectral clustering is used. Such speaker changes can result from temporary or permanent changes in vocal tract characteristics or from environmental effects. Thus, the codebook performance is improved by collecting heart sound patterns over a long period of time to account for natural variations in speaker behavior. In one embodiment, data from the vector quantizer is presented to one or more recognition models, including an HMM model, a dynamic time warping model, a neural network, a fuzzy logic, or a template matcher, among others. These models may be used singly or in combination.

In dynamic processing, at the time of recognition, dynamic programming slides, or expands and contracts, an operating region, or window, relative to the frames of heart sound so as to align those frames with the node models of each S1-S4 pattern to find a relatively optimal time alignment between those frames and those nodes. The dynamic processing in effect calculates the probability that a given sequence of frames matches a given word model as a function of how well each such frame matches the node model with which it has been time-aligned. The word model which has the highest probability score is selected as corresponding to the heart sound.

Dynamic programming obtains a relatively optimal time alignment between the heart sound to be recognized and the nodes of each word model, which compensates for the unavoidable differences in speaking rates which occur in different utterances of the same word. In addition, since dynamic programming scores words as a function of the fit between word models and the heart sound over many frames, it usually gives the correct word the best score, even if the word has been slightly misspoken or obscured by background sound. This is important, because humans often mispronounce words either by deleting or mispronouncing proper sounds, or by inserting sounds which do not belong.

In another aspect, a monitoring system for a person includes one or more wireless bases; and a cellular telephone having a wireless transceiver adapted to communicate with the one or more wireless bases; and an accelerometer to detect a dangerous condition and to generate a warning when the dangerous condition is detected. In yet another aspect, a monitoring system includes one or more cameras to determine a three dimensional (3D) model of a person; means to detect a dangerous condition based on the 3D model; and means to generate a warning when the dangerous condition is detected. In another aspect, a method to detect a dangerous condition for an infant includes placing a pad with one or more sensors in the infant's diaper; collecting infant vital parameters; processing the vital parameter to detect SIDS onset; and generating a warning.

AI or machine learning can be applied to the data collected by the sensors. The processing can be done by a remote computer or can be done using the local CPU. For example, a Markov model is formed for a reference pattern from a plurality of sequences of training patterns and the output symbol probabilities are multivariate Gaussian function probability densities. The patient habit information is processed by a feature extractor. During learning, the resulting feature vector series is processed by a parameter estimator, whose output is provided to the hidden Markov model. The hidden Markov model is used to derive a set of reference pattern templates, each template representative of an identified pattern in a vocabulary set of reference treatment patterns. The Markov model reference templates are next utilized to classify a sequence of observations into one of the reference patterns based on the probability of generating the observations from each Markov model reference pattern template. During recognition, the unknown pattern can then be identified as the reference pattern with the highest probability in the likelihood calculator. The HMM template has a number of states, each having a discrete value. However, because treatment pattern features may have a dynamic pattern in contrast to a single value. The addition of a neural network at the front end of the HMM in an embodiment provides the capability of representing states with dynamic values. The input layer of the neural network comprises input neurons. The outputs of the input layer are distributed to all neurons in the middle layer. Similarly, the outputs of the middle layer are distributed to all output states, which normally would be the output layer of the neuron. However, each output has transition probabilities to itself or to the next outputs, thus forming a modified HMM. Each state of the thus formed HMM is capable of responding to a particular dynamic signal, resulting in a more robust HMM. Alternatively, the neural network can be used alone without resorting to the transition probabilities of the HMM architecture.

Power generation with piezoelectrics can be done with body vibrations or by physical compression (impacting the material and using a rapid deceleration using foot action, for example). The vibration energy harvester consists of three main parts. A piezoelectric transducer (PZT) serves as the energy conversion device, a specialized power converter rectifies the resulting voltage, and a capacitor or battery stores the power. The PZT takes the form of an aluminum cantilever with a piezoelectric patch. The vibration-induced strain in the PZT produces an ac voltage. The system repeatedly charges a battery or capacitor, which then operates the EKG/EMG sensors or other sensors at a relatively low duty cycle. In one embodiment, a vest made of piezoelectric materials can be wrapped around a person's chest to generate power when strained through breathing as breathing increases the circumference of the chest for an average human by about 2.5 to 5 cm. Energy can be constantly harvested because breathing is a constant activity, even when a person is sedate. When the stave is bent, the piezoelectric sheets on the outside surface are pulled into expansion, while those on the inside surface are pushed into contraction due to their differing radii of curvature, producing voltages across the electrodes. In another embodiment, PZT materials from Advanced Cerametrics, Inc., Lambertville, N.J. can be incorporated into flexible, motion sensitive (vibration, compression or flexure), active fiber composite shapes that can be placed in shoes, boots, and clothing or any location where there is a source of waste energy or mechanical force. These flexible composites generate power from the scavenged energy and harness it using microprocessor controls developed specifically for this purpose. Advanced Cerametric's viscose suspension spinning process (VSSP) can produce fibers ranging in diameter from 10 μm ( 1/50 of a human hair) to 250 μm and mechanical to electrical transduction efficiency can reach 70 percent compared with the 16-18 percent common to solar energy conversion. The composite fibers can be molded into user-defined shapes and is flexible and motion-sensitive. In one implementation, energy is harvested by the body motion such as the foot action or vibration of the PZT composites. The energy is converted and stored in a low-leakage charge circuit until a predetermined threshold voltage is reached. Once the threshold is reached, the regulated power may flow for a sufficient period to power the wireless node such as the Zigbee CPU/transceiver. The transmission is detected by nearby wireless nodes that are AC-powered and forwarded to the base station for signal processing. Power comes from the vibration of the system being monitored and the unit requires no maintenance, thus reducing lifecycle costs. In one embodiment, the housing of the unit can be PZT composite, thus reducing the weight.

In another embodiment, body energy generation systems include electro active polymers (EAPs) and dielectric elastomers. EAPs are a class of active materials that have a mechanical response to electrical stimulation and produce an electric potential in response to mechanical stimulation. EAPs are divided into two categories, electronic, driven by electric field, and ionic, driven by diffusion of ions. In one embodiment, ionic polymers are used as biological actuators that assist muscles for organs such as the heart and eyes. Since the ionic polymers require a solvent, the hydrated human body provides a natural environment. Polymers are actuated to contract, assisting the heart to pump, or correcting the shape of the eye to improve vision. Another use is as miniature surgical tools that can be inserted inside the body. EAPs can also be used as artificial smooth muscles, one of the original ideas for EAPs. These muscles could be placed in exoskeletal suits for soldiers or prosthetic devices for disabled persons. Along with the energy generation device, ionic polymers can be the energy storage vessel for harvesting energy. The capacitive characteristics of the EAP allow the polymers to be used in place of a standard capacitor bank. With EAP based jacket, when a person moves his/her arms, it will put the electro active material around the elbow in tension to generate power. Dielectric elastomers can support 50-100% area strain and generate power when compressed. Although the material could again be used in a bending arm type application, a shoe type electric generator can be deployed by placing the dielectric elastomers in the sole of a shoe. The constant compressive force provided by the feet while walking would ensure adequate power generation.

For wireless nodes that require more power, electromagnetics, including coils, magnets, and a resonant beam, and micro-generators can be used to produce electricity from readily available foot movement. Typically, a transmitter needs about 30 mW, but the device transmits for only tens of milliseconds, and a capacitor in the circuit can be charged using harvested energy and the capacitor energy drives the wireless transmission, which is the heaviest power requirement. Electromagnetic energy harvesting uses a magnetic field to convert mechanical energy to electrical. A coil attached to the oscillating mass traverses through a magnetic field that is established by a stationary magnet. The coil travels through a varying amount of magnetic flux, inducing a voltage according to Faraday's law. The induced voltage is inherently small and must therefore be increased to viably source energy. Methods to increase the induced voltage include using a transformer, increasing the number of turns of the coil, and/or increasing the permanent magnetic field. Electromagnetic devices use the motion of a magnet relative to a wire coil to generate an electric voltage. A permanent magnet is placed inside a wound coil. As the magnet is moved through the coil it causes a changing magnetic flux. This flux is responsible for generating the voltage which collects on the coil terminals. This voltage can then be supplied to an electrical load. Because an electromagnetic device needs a magnet to be sliding through the coil to produce voltage, energy harvesting through vibrations is an ideal application. In one embodiment, electromagnetic devices are placed inside the heel of a shoe. One implementation uses a sliding magnet-coil design, the other, opposing magnets with one fixed and one free to move inside the coil. If the length of the coil is increased, which increases the turns, the device is able to produce more power.

In an electrostatic (capacitive) embodiment, energy harvesting relies on the changing capacitance of vibration-dependant varactors. A varactor, or variable capacitor, is initially charged and, as its plates separate because of vibrations, mechanical energy is transformed into electrical energy. MEMS variable capacitors are fabricated through relatively mature silicon micro-machining techniques.

In another embodiment, the wireless node can be powered from thermal and/or kinetic energy. Temperature differentials between opposite segments of a conducting material result in heat flow and consequently charge flow, since mobile, high-energy carriers diffuse from high to low concentration regions. Thermopiles consisting of n- and p-type materials electrically joined at the high-temperature junction are therefore constructed, allowing heat flow to carry the dominant charge carriers of each material to the low temperature end, establishing in the process a voltage difference across the base electrodes. The generated voltage and power is proportional to the temperature differential and the Seebeck coefficient of the thermoelectric materials. Body heat from a user's wrist is captured by a thermoelectric element whose output is boosted and used to charge the a lithium ion rechargeable battery. The unit utilizes the Seeback Effect which describes the voltage created when a temperature difference exists across two different metals. The thermoelectric generator takes body heat and dissipates it to the ambient air, creating electricity in the process.

In another embodiment, the kinetic energy of a person's movement is converted into energy. As a person moves their weight, a small weight inside the wireless node moves like a pendulum and turns a magnet to produce electricity which can be stored in a super-capacitor or a rechargeable lithium battery. Similarly, in a vibration energy embodiment, energy extraction from vibrations is based on the movement of a “spring-mounted” mass relative to its support frame. Mechanical acceleration is produced by vibrations that in turn cause the mass component to move and oscillate (kinetic energy). This relative displacement causes opposing frictional and damping forces to be exerted against the mass, thereby reducing and eventually extinguishing the oscillations. The damping forces literally absorb the kinetic energy of the initial vibration. This energy can be converted into electrical energy via an electric field (electrostatic), magnetic field (electromagnetic), or strain on a piezoelectric material.

Another embodiment extracts energy from the surrounding environment using a small rectenna (microwave-power receivers or ultrasound power receivers) placed in patches or membranes on the skin or alternatively injected underneath the skin.

The rectanna converts the received emitted power back to usable low frequency/dc power. A basic rectanna consists of an antenna, a low pass filter, an ac/dc converter and a dc bypass filter. The rectanna can capture renewable electromagnetic energy available in the radio frequency (RF) bands such as AM radio, FM radio, TV, very high frequency (VHF), ultra high frequency (UHF), global system for mobile communications (GSM), digital cellular systems (DCS) and especially the personal communication system (PCS) bands, and unlicensed ISM bands such as 2.4 GHz and 5.8 GHz bands, among others. The system captures the ubiquitous electromagnetic energy (ambient RF noise and signals) opportunistically present in the environment and transforming that energy into useful electrical power. The energy-harvesting antenna is preferably designed to be a wideband, omnidirectional antenna or antenna array that has maximum efficiency at selected bands of frequencies containing the highest energy levels. In a system with an array of antennas, each antenna in the array can be designed to have maximum efficiency at the same or different bands of frequency from one another. The collected RF energy is then converted into usable DC power using a diode-type or other suitable rectifier. This power may be used to drive, for example, an amplifier/filter module connected to a second antenna system that is optimized for a particular frequency and application. One antenna system can act as an energy harvester while the other antenna acts as a signal transmitter/receiver. The antenna circuit elements are formed using standard wafer manufacturing techniques. The antenna output is stepped up and rectified before presented to a trickle charger. The charger can recharge a complete battery by providing a larger potential difference between terminals and more power for charging during a period of time. If battery includes individual micro-battery cells, the trickle charger provides smaller amounts of power to each individual battery cell, with the charging proceeding on a cell by cell basis. Charging of the battery cells continues whenever ambient power is available. As the load depletes cells, depleted cells are switched out with charged cells. The rotation of depleted cells and charged cells continues as required. Energy is banked and managed on a micro-cell basis.

In a solar cell embodiment, photovoltaic cells convert incident light into electrical energy. Each cell consists of a reverse biased pn+ junction, where light interfaces with the heavily doped and narrow n+ region. Photons are absorbed within the depletion region, generating electron-hole pairs. The built-in electric field of the junction immediately separates each pair, accumulating electrons and holes in the n+ and p-regions, respectively, and establishing in the process an open circuit voltage. With a load connected, accumulated electrons travel through the load and recombine with holes at the p-side, generating a photocurrent that is directly proportional to light intensity and independent of cell voltage.

As the energy-harvesting sources supply energy in irregular, random “bursts,” an intermittent charger waits until sufficient energy is accumulated in a specially designed transitional storage such as a capacitor before attempting to transfer it to the storage device, lithium-ion battery, in this case. Moreover, the system must partition its functions into time slices (time-division multiplex), ensuring enough energy is harvested and stored in the battery before engaging in power-sensitive tasks. Energy can be stored using a secondary (rechargeable) battery and/or a supercapacitor. The different characteristics of batteries and supercapacitors make them suitable for different functions of energy storage. Supercapacitors provide the most volumetrically efficient approach to meeting high power pulsed loads. If the energy must be stored for a long time, and released slowly, for example as back up, a battery would be the preferred energy storage device. If the energy must be delivered quickly, as in a pulse for RF communications, but long term storage is not critical, a supercapacitor would be sufficient. The system can employ i) a battery (or several batteries), ii) a supercapacitor (or supercapacitors), or iii) a combination of batteries and supercapacitors appropriate for the application of interest. In one embodiment, a microbattery and a microsupercapacitor can be used to store energy. Like batteries, supercapacitors are electrochemical devices; however, rather than generating a voltage from a chemical reaction, supercapacitors store energy by separating charged species in an electrolyte. In one embodiment, a flexible, thin-film, rechargeable battery from Cymbet Corp. of Elk River, Minn. provides 3.6V and can be recharged by a reader. The battery cells can be from 5 to 25 microns thick. The batteries can be recharged with solar energy, or can be recharged by inductive coupling. The tag is put within range of a coil attached to an energy source. The coil “couples” with the antenna on the RFID tag, enabling the tag to draw energy from the magnetic field created by the two coils.

One embodiment provides a nasal bone conduction wireless communication transmitting device in or near the nostril. The bone conduction includes a carrier provided on or in the nose for making a bone conduction outputting device and the bone conduction inputting device supported by it closely touching the skin of the nasal bone. The system makes the oscillating wave of the bone conduction outputting device being sent to the ear via the nasal bone conduction after the resonance in a nasal cavity; and converting the sound provided through the resonance in the nasal cavity by the bone conduction inputting device into an electrical wave, and then transmitting it to the wireless communication transmitting device for signaling.

The signal processing unit of the abovementioned wireless communication transmitting device is used for processing a signal, and the signal processing unit has a signal processor, a default parameter value storage, a parameter modification register, a function module, and a feedback elimination processing module.

In another aspect, a method for fabricating a custom eye wear device with built-in air filter and health sensor is disclosed. The method includes scanning 3D dimensions of a face, generating an eyeglass frame covering a nose or a mouth based on the 3D dimensions of the face, generating a frame with air duct paths to a nose or a mouth, and 3D printing an eyeglass frame customized to the face.

The method further comprises 3D printing antennas suitable for 5G cellular communication on the surface of the frame and connecting the antennas to transceivers, which in turn is connected to sensors and processors. The sensors can be flexible printed electronics, and detailed in Application Publication 20200008299 to Bao Tran, the content of which is incorporated by reference.

The eye wear system of FIG. 2A allows the user to enjoy AR/VR/XR contents in a protected environment while exploring the world. In one aspect, Systems and methods are disclosed for recommending products or services by receiving a 3D model of a product; capturing a reference object with a predetermined dimension in an environment where the product is to be placed using a mobile camera; determining one more dimensions of the environment relative to the predetermined dimension of the reference object; scaling the 3D model of the product based on dimensions of the environment and the product; and generating an augmented or virtual reality display of the product in the environment.

In implementations, the reference object can be a coin or a sheet of paper with predetermined dimensions. The mobile camera can be a smart phone or a portable camera. The camera can be an infrared camera. The product can be an appliance or furniture, a wearable item, a jean, or a shirt. For clothing, the system can render an image of the object on a mannequin. The system can monitor user health by analyzing changes in the 3D model over time. The system can analyze a user anatomical portion and selecting a best fit from apparel variations. The product can be cosmetic product, a facial makeup product, or a hair product. The system includes motion tracking, area learning and depth sensing the product. The system can create a 3D model using infrared images. The system includes identifying one or more best fitting products to the environment and displaying recommendations with one or more best fitting products in the environment. The best fitting products can be clothing, shoes, cosmetics, appliances or furniture. The method includes capturing 3D model of user's feet; identify the subject's current best fitting shoe products; set each best fitting shoe product's inside dimension with dimensions from the 3D model plus a predetermined gap; correlating different manufacturer's shoe sizes and creating correspondences among different manufacturer shoe products; and recommending a new shoe for the subject by looking up the correspondences among different manufacturer shoe products.

In another aspect, a method for best fitting product variations to an environment by receiving a 3D model of a product with one or more product variations; capturing a reference object with a predetermined dimension in an environment where the product is to be placed using a mobile camera; determining one more dimensions of the environment relative to the predetermined dimension of the reference object; scaling the 3D model of each product variation based on dimensions of the environment and characteristics of the product variation; and generating an augmented or virtual reality display of the product in the environment.

In yet another aspect, a method for recommending a service includes receiving a model of a service to be applied to a target object; capturing a reference object with a predetermined dimension in an environment where the service is to be applied to the target object using a mobile camera; determining one more dimensions of the environment relative to the predetermined dimension of the reference object; generating a 3D model of the service as applied to the target object; scaling the 3D model of the generated 3D model based on dimensions of the environment and the product; and generating an augmented or virtual reality display of the product in the environment.

In implementations, the service to a product can be for one of: a cosmetic product, a plastic surgery medical device, a facial makeup product, a hair product. For example, for make up, the method includes capturing images of a face and a reference object from a plurality of angles using a mobile camera; creating a 3D model of the face from the images with dimensions based on dimensions of the reference object; selecting a makeup pattern or color from a plurality of makeup product variations; and blending the makeup pattern or color onto the 3D model; and displaying the makeup color on the face. If the target object is a breast implant, the method includes recommending a breast augmentation sizing to a patient. In another aspect, a camera tracks movements and a 3-D scanner analyzes the viewer's physique. Body recognition software analyzes the body shape to determine weight loss or gain. In addition to shoe/clothing suggestions, the system can provide clothing/jewelry/hair styling suggestions along with augmented reality view of the suggestions so that the user can visualize the impact of the clothing or jewelry or styling. Facial recognition software inspects the face shape to determine health. The smart mirror can provide make-up suggestions along with augmented reality view of the applied suggestions so that the user can visualize the impact of the makeup. The smart mirror can provide non-surgical body augmentation suggestions such as breast/buttock augmentations along with augmented reality view of the body enlargements or size reduction so that the user can visualize the impact of the footwear or apparel when worn, along with body enhancement, clothing or jewelry or hair styling changes. In yet another aspect, built-in sensors in combination with mobile phone usage pattern and social network communications can detect signs of stress and other mental/emotional health states of the user. The smart insole or shoes with sensors could also be combined with other health-related apps to keep track of calorie count, vital signs, fitness level and sleep quality. By extrapolating from the user's current behaviors, vitals and bone and muscle structure, the augmented-reality mirror can forecast the user's future health. The camera can measure breathing activity and/or heart rate of the user in front of the mirror or alternatively the system can bounce WiFi off the chest to detect breathing activity. The mirror highlights hard-to-see changes in the body, such as increased fatigue, minute metabolic imbalances and more. A DNA analyzer can receive swipes from tongue, ear, and saliva, bodily fluids to capture genetic data at a high frequency and such data can be correlated with the fitness wearable devices for signs of health problems. Additionally, the data can be analyzed at a metropolitan level for public health purposes.

The methods presently disclosed may provide a healthcare provider with a more complete record of a user's day-to-day status. By having access to a consistent data stream of breathing and air quality metrics for a user, a healthcare provider may be able to provide the user with timely advice and real-time coaching on corona virus infection management options and solutions. A user may, for example, seek and/or receive feedback on without waiting for an upcoming appointment with a healthcare provider or scheduling a new appointment. Such real-time communication capability may be especially beneficial to provide users with guidance and treatment options during intervals between appointments with a healthcare provider. Healthcare providers may also be able to monitor a user's status between appointments to timely initiate, modify, or terminate a treatment plan as necessary. For example, a user's reported medication use may convey whether the user is taking too little or too much medication. In some embodiments, an alert may be triggered to notify the user and/or a healthcare provider of the amount of medication taken, e.g., in comparison to a prescribed treatment plan. The healthcare provider could, for example, contact the user to discuss the treatment plan. The methods disclosed herein may also provide a healthcare provider with a longitudinal review of how a user responds to pathogen or infection treatment over time. For example, a healthcare provider may be able to determine whether a given treatment plan adequately addresses a user's needs based on review of the user's reported metrics and analysis thereof according to the present disclosure.

Analysis of user data according to the methods presently disclosed may generate one or more recommended actions that may be transmitted and displayed on an output device. In some embodiments, the analysis recommends that a user make no changes to his/her treatment plan or routine. In other embodiments, the analysis generates a recommendation that the user seek further consultation with a healthcare provider and/or establish compliance with a prescribed treatment plan. In other embodiments, the analysis may encourage a user to seek immediate medical attention. For example, the analysis may generate an alert to be transmitted to one or more output devices, e.g., a first output device belonging to the user and a second output device belonging to a healthcare provider, indicating that the user needs immediate medical treatment. In some embodiments, the analysis may not generate a recommended action. Other recommended actions consistent with the present disclosure may be contemplated and suitable according to the treatment plans, needs, and/or preferences for a given user.

The present disclosure further provides a means for monitoring a user's medication use to determine when his/her prescription will run out and require a refill. For example, a user profile may be created that indicates a prescribed dosage and frequency of administration, as well as total number of dosages provided in a single prescription. As the user reports medication use, those infectious treatment metrics may be transmitted to a server and stored in a database in connection with the user profile. The user profile stored on the database may thus continually update with each added metric and generate a notification to indicate when the prescription will run out based on the reported medication use. The notification may be transmitted and displayed on one or more output devices, e.g., to a user and/or one or more healthcare providers. In some embodiments, the one or more healthcare providers may include a pharmacist. For example, a pharmacist may receive notification of the anticipated date a prescription will run out in order to ensure that the prescription may be timely refilled.

User data can be input for analysis according to the systems disclosed herein through any data-enabled device including, but not limited to, portable/mobile and stationary communication devices, and portable/mobile and stationary computing devices. Non-limiting examples of input devices suitable for the systems disclosed herein include smart phones, cell phones, laptop computers, netbooks, personal computers (PCs), tablet PCs, fax machines, personal digital assistants, and/or personal medical devices. The user interface of the input device may be web-based, such as a web page, or may also be a stand-alone application. Input devices may provide access to software applications via mobile and wireless platforms, and may also include web-based applications.

The input device may receive data by having a user, including, but not limited to, a user, family member, friend, guardian, representative, healthcare provider, and/or caregiver, enter particular information via a user interface, such as by typing and/or speaking. In some embodiments, a server may send a request for particular information to be entered by the user via an input device. For example, an input device may prompt a user to enter sequentially a set of infectious treatment metrics, e.g., a infectious treatment score, a functionality score, and information regarding use of one or more medications (e.g., type of medication, dosage taken, time of day, route of administration, etc.). In other embodiments, the user may enter data into the input device without first receiving a prompt. For example, the user may initiate an application or web-based software program and select an option to enter one or more infectious treatment metrics. In some embodiments, one or more infectious treatment scales and/or functionality scales may be preselected by the application or software program. For example, a user may have the option of selecting the type of infectious treatment scale and/or functionality scale for reporting infectious treatment metrics within the application or software program. In other embodiments, an application or software program may not include preselected infectious treatment scales or functionality scales such that a user can employ any infectious treatment scale and/or functionality scale of choice.

In exemplary system for mining health data for precision medicine, medical grade data from the user's physician/hospital, along with 3D models, and lab test equipment data are stored in a database. Omic test equipment also generates data that is stored in another database. EHR data from primary care physician (PHP), emergency room physicians (ER), and in-patient care data is also stored in a database. These databases form a clinical data repository that contains medical diagnosis and treatment information. The clinical data is high grade medical information that is secured by patient privacy laws such as HIPPA. One exemplary process for improving healthcare using precision medicine includes:

obtain clinical data from mirror and 3d party laboratory test equipment

obtain clinical data from one or more omic test equipment

obtain clinical data from a primary care physician database

obtain clinical data from a specialist physician database

obtain clinical data from an emergency room database

obtain clinical data from an in-patient care database

save the clinical data into a clinical data repository

obtain health data from fitness devices and from mobile phones

obtain behavioral data from social network communications and mobile device usage patterns

save the health data and behavioral data into a health data repository separate from the clinical data repository

mine the clinical data repository and health data repository for patients sharing similarity with the subject, including one or more similar biomarkers associated with health conditions

identify at least one similar health conditions and identifying one or more corrective actions recorded in the repository and the result of each action for the one or more health conditions;

present the corrective action and result to the subject and recommending an action to reduce risk from the predicted health condition

monitor the health condition using updates in the clinical data repository and health data repository

In another embodiment for cost effective health maintenance, the system includes a method of insuring a subject for cancer, by:

enrolling the subject into a cost-saving program;

receiving a body sample during routine periodic examinations and characterizing the subject's omic information with a DNA sequencer; and

using historical omic information to detect an occurrence of a disease such as cancer before the subject is suspected of having the disease; and

proactively recommending early treatments based on the omic information received at each time interval to cost-effectively control disease.

The system processes a communication from a patient according to one or more treatment scenarios. Each treatment scenario is composed of one or more rules to be processed in a sequence that can be altered when invoking certain agents.

The if then rules can be described to the system using a graphical user interface that runs on a web site, a computer, or a mobile device, and the resulting rules are then processed by a rules engine. In one embodiment, the if then rules are entered as a series of dropdown selectors whose possible values are automatically determined and populated for user selection to assist user in accurately specifying the rules.

One embodiment determines high interest disease- and drug-related variants in the pateint's genome and identifies top diseases with the highest probabilities. For each disease, the system determines the pretest probability according to the patient age, gender, and ethnicity. The system then determines the independent disease-associated SNVs used to calculate the subject's disease probability. For each disease, for example type 2 Covid or infectious disease, the system determines probability using independent SNVs, a likelihood ratio (LR), number of studies, cohort sizes, and the posttest probability. Blood pressure and blood glucose trend measurements are also determined.

In an exemplary system for providing precision medicine, historical data from a large population is received and provided to a learning engine. The learning engine clusters the population into groups of similar characteristics and then creates a social network of patients who share enough health/medical similarity that they are apt to share many medical issues. Thus a user's likelihood of contracting a disease might be evaluated by knowing the disease status of other users in the same influence cluster or neighborhood, whether they are closely connected to that user or not. Contact tracing network data can be used to determine population level health in case of an epidemic.

The system, generally denoted by reference numeral 100, comprises one or more central processing units CP1 . . . CPn, generally denoted by reference numeral 110. Embodiments comprising multiple processing units 110 are preferably provided with a load balancing unit 115 that balances processing load among the multiple processing units 110. The multiple processing units 110 may be implemented as separate processor components or as physical processor cores or virtual processors within a single component case. In a typical implementation the computer architecture 100 comprises a network interface 120 for communicating with various data networks, which are generally denoted by reference sign DN. The data networks DN may include local-area networks, such as an Ethernet network, and/or wide-area networks, such as the internet. In some implementations the computer architecture may comprise a wireless network interface, generally denoted by reference numeral 125. By means of the wireless network interface, the computer 100 may communicate with various access networks AN, such as cellular networks or Wireless Local-Area Networks (WLAN). Other forms of wireless communications include short-range wireless techniques, such as Bluetooth and various “Bee” interfaces, such as XBee, ZigBee or one of their proprietary implementations. Depending on implementation, a user interface 140 may comprise local input-output circuitry for a local user interface, such as a keyboard, mouse and display (not shown). The computer architecture also comprises memory 150 for storing program instructions, operating parameters and variables. Reference numeral 160 denotes a program suite for the server computer 100. Reference number 115-135 denotes an optional interface by which the computer obtains data from external sensors, analysis equipment or the like.

In some embodiments the data processing system is coupled with equipment that determines an organism's genotype from an in-vitro sample obtained from the organism. In other embodiments the genotypes are determined elsewhere and the data processing system may obtain data representative of the genotype via any of its data interfaces.

One exemplary sensor communicating with one of the interfaces 115-135 receives a biologic sample from an individual such as a bodily fluid (such as urine, saliva, plasma, or serum) or feces or a tissue sample (such as a buccal tissue sample or buccal cell). The biologic sample can then be used to perform a genome scan. For example, DNA arrays can be used to analyze at least a portion of the genomic sequence of the individual. Exemplary DNA arrays include GeneChip Arrays, GenFlex Tag arrays, and Genome-Wide Human SNP Array 6.0 (available from Affymetrix, Santa Clara, Calif.). In other examples, DNA sequencing with commercially available next generation sequencing (NGS) platforms is generally conducted: DNA sequencing libraries are generated by clonal amplification by PCR in vitro; then the DNA is sequenced by synthesis, such that the DNA sequence is determined by the addition of nucleotides to the complementary strand rather through chain-termination chemistry; next, the spatially segregated, amplified DNA templates are sequenced simultaneously in a massively parallel fashion without the requirement for a physical separation step. For microbiome analysis, cotton swabs are applied to forehead, behind ears, nose, among others, and fecal samples are analyzed using DNA sequencing machines. In certain embodiments, whole or partial genome sequence information is used to perform the genome scans. Such sequences can be determined using standard sequencing methods including chain-termination (Sanger dideoxynucleotide), dye-terminator sequencing, and SOLiD™ sequencing (Applied Biosystems). Whole genome sequences can be cut by restriction enzymes or sheared (mechanically) into shorter fragments for sequencing. DNA sequences can also be amplified using known methods such as PCR and vector-based cloning methods (e.g., Escherichia coli).

The sensors connecting to interfaces 115-135 can also include fitness sensors such as wearable watches/clothing/shoes that monitor activity, heart rate, ECG, blood pressure, blood oxygen level, among others. The sensors 115-135 can also detect purchase activities and on-line activities that reflect the user's health habits. For example, the sensors can be a data feed that picks up data relating to grocery purchases, food expenses, restaurant spending.

In yet other examples, the sensors connecting to interfaces 115-135 can be sensors in a phone. For example, in depression sensor, the phone can detect a person's activity and correlate to depression: people who stuck to a regular pattern of movement tended to be less depressed as people with mental health problems in general have disrupted circadian rhythms and a depressed mood may pull a user off her routine. Depressed people also spends more time on their phones or browsing aimlessly, as depressed people tend to start avoiding tasks or things they have to do, particularly when they're uncertain.

In addition to sensor captured healthcare data, healthcare data refers to any data related or relevant to a patient. Healthcare data may include, but is not limited to, fitness data and healthcare-related financial data. Clinical data, as used herein, refers to any healthcare or medical data particular to a patient. In embodiments, clinical data can be medical care or healthcare data resulting from or associated with a health or medical service performed in association with a clinician in a healthcare environment (e.g., lab test, diagnostic test, clinical encounter, ecare, evisit, etc.). Clinical data may include, but is not limited to, a health history of a patient, a diagnosis, a clinician assessment, clinician narrative, a treatment, a family history (including family health history and/or family genetics), an immunization record, a medication, age, gender, date of birth, laboratory values, diagnostics, a test result, an allergy, a reaction, a procedure performed, a social history, an advanced directive, frequency and/or history of healthcare facility visits, current healthcare providers and/or current healthcare provider location, preferred pharmacy, prescription benefit management data, an alert, claims data, a vital, data traditionally captured at the point of care or during the care process, a combination thereof, and the like. In the same or alternative embodiments, the clinical data may include medical compliance information. In certain embodiments, medical compliance information refers to a level of compliance of a particular patient with one or more prescribed medical treatments, such as medications, diet, physical therapy, follow up healthcare visits, and the like. In one or more embodiments, the clinical data may include data obtained from the natural language processing of one or more clinical assessments and/or clinical narratives.

By engaging and empowering patients to take an active role in data collection, the footwear applies inconspicuous foot data with analytics to improve health. One embodiment uses Google Maps to display health activity traffic; showing healthcare patterns based on real time reporting of anonymous data from healthcare footware devices. Healthcare organizations can tap the power of that data to engage patients and develop more effective and more personalized approaches to care, thereby lowering the overall cost of care.

The system identifies pre-detectable characteristics of a health condition, such that future incidents of the health condition may be predicted, i.e., before the health condition occurs for disease prevention. One implementation includes capturing data from mobile fitness devices and establishing a plurality of health related characteristics associated with the population including walking status, weight, calorie burn. The characteristics include a plurality of pre-detectable characteristics with a relationship between the health related characteristics and at least one health condition, and analyzing at least a portion of said population in response to the relationship.

Another embodiment includes establishing at least one pre-detectable characteristic associated with a health condition, applying an intervention in response to the characteristic, monitoring a success characteristic of the intervention, and determining a cause of the success characteristic.

Another embodiment builds a repository of health related characteristics associated with the population, the characteristics including a plurality of pre-detectable characteristics; and a processor configured to receive the health related characteristics, establish a relationship between the health related characteristics and at least one health condition, and analyzing at least a portion of the population in response to said relationship.

A population, as used herein, is any group of members. The population may include a high level of members, for example a group including one or more of the five kingdoms of living things, or a subgroup, for example a group including humans of a certain age range. The population may include living and/or dead members. The analysis may include predicting a likelihood of a member developing the health condition, in response to the relationship. The health condition may be any type of physical or mental health condition, disease, and/or ailment. In addition, the analysis may include predicting the incidence of the health condition. The analysis may also include performing a simple yes/no prediction regarding whether a member will likely develop the health condition. The analysis may be used to enable the management of a health care program, such as a program associated with a corporation, or a program offered to the public by a health care consultant or provider. If the analysis is associated with a corporation's healthcare program, the population may include some or all of the employees and retirees of the corporation, and associated spouses and dependents. The population may include other associated groups of the corporation, such as consultants, contractors, suppliers and/or dealers. The population may include participants from multiple corporations and/or the general public. If the health care program is offered to the public, the population may include members of the public, organizations, and/or corporations.

The health related characteristics may include a plurality of health characteristics, lifestyle characteristics and/or family health characteristics associated with the members of the population. Health characteristics may include characteristics indicative of a specific member's health. For example, lifestyle characteristic may include weight, heart rate, walking gait, sitting gait, running gait, exercise or activity as detected by accelerometers, diet, and other factors detectable by fitness devices such as watches, phones, or foot sensors detailed above. For other example, health characteristic may include medical characteristics (e.g., what medical visits, processes, procedures, or test have been performed associated with the member, the number of days the member has spent in a medical facility (e.g., a hospital), the number of visits the person has made to a doctor, etc.), drug characteristics (e.g., what type and amount of drugs are being consumed), a death characteristic (e.g., information associated with a death certificate), an absenteeism characteristic, disability characteristics, characteristics associated with existing health conditions, etc. Family health characteristics associated with the member may include information associated with the family medical history of a specific member. For example, a history of a particular health risk within the family, e.g., heart failure, cancer, high blood pressure, Covid or infectious disease, anxiety, stress, etc. Lifestyle characteristic may include a specific member's behavior characteristic(s), of which some or all may be modifiable lifestyle characteristics. A modifiable lifestyle characteristic may include an exercise characteristic (e.g., does the member exercise, how often, what is the exercise, etc.) and/or a nutrition characteristic (e.g., what types of food does the member eat, and how often). Nutrition characteristics may also include the amount of salt consumed during a designated period (e.g., a day), and the amount of fat and/or saturated fat consumed during a designated period. In addition, modifiable lifestyle characteristics may include whether the member drinks alcohol (and if so how much), a drug intake characteristic, (i.e., does the member take drugs, and if so how often, what kind, and how much), a weight characteristic (e.g., what does the member weigh, what is the member's desired weight, is the member on a diet, what is the member's weight indicator e.g., obese, slightly overweight, underweight, normal, etc.), a smoking characteristic (does the member smoke and if so how much), a safety characteristic (what are the member's driving characteristics e.g., does the member where seat belts, have one or more infractions associated with driving under the influence, or speeding tickets, etc.). In addition, modifiable lifestyle characteristics may include a infectious treatment characteristic, a stress characteristic, a self-care characteristic, a self-efficacy characteristic, a readiness to change characteristics, and a prophylactic aspirin therapy characteristic.

One method for performing population health management includes establishing a plurality of health related characteristics associated with the population; establishing a relationship between the health related characteristics and at least one health condition; and analyzing at least a portion of said population in response to said relationship. The system can predict a likelihood of at least one of said members developing said at least one health condition, in response to said relationship and/or the members health related characteristics. The system can determine a prevalence of a health condition within said population in response to said health related characteristics. The plurality of health related characteristics associated with said population can be done by establishing a plurality of self-reported characteristics associated with at least a portion of said population. A prevalence of the health condition can be determined by: establishing a plurality of claims associated with at least one os said members, said claims including at least one of a drug claim and a medical claim; cross checking said plurality of claims (such as over a period of time, or over a number of tests); and establishing said prevalence in response to said cross checked claims. The system includes predicting a member's likelihood of developing a condition with a stage of said condition in response to said prediction. The system can predict a time period associated with said development. The system can classify said population in response to said prediction, and then prioritize treatment of the population in response to said prediction.

The system can recommend an intervention in response to said predicted likelihood of development. This can be done by establishing a plurality of intervention recommendations associated with said condition; establishing a success characteristics of said recommended intervention; establishing at least one of a readiness to change characteristic and a self-efficacy characteristic of said member; and recommending said intervention in response to said plurality of intervention recommendations, associated intervention success characteristics, and member health related characteristics, said health characteristics including said self-efficacy and said readiness to change characteristic.

The system can monitor failure/successful characteristic of said intervention, and determining causes resulting in said success characteristic. The system can capture a plurality of self-reported data associated with at least a portion of said population having said condition. The self-reported data includes at least one of a lifestyle characteristic, a family history characteristic, and a health characteristic. The predictive relationship can be done by establishing at least one objective of said relationship; dynamically selecting a statistical analysis technique in response to said objective; and establishing said relationship in response to said statistical analysis technique. The predictive relationship can be applied to at least a portion of said population; and predicting a likelihood of developing said condition in response to said application.

The system can be configured to analyze the health of a population having multiple members. In one embodiment, the method includes the steps of establishing a plurality of health related characteristics associated with the population, the characteristics including a plurality of pre-detectable characteristics, establishing a relationship between the health related characteristics and the health condition, and predicting an incident of the health condition associated with at least one of the members, in response to the relationship. The health condition may be any type of physical or mental health condition, disease, and/or ailment. For exemplary purposes the method and system will be discussed as they may relate to the health condition Covid or infectious disease. A repository of health related characteristics associated with a population may be collected. The health related characteristics may be collected through sources such as medical claims, drug claims, and self-reported information. The characteristics may include health characteristics, lifestyle characteristics, and family history characteristics. The characteristics may include the amount of saturated fat, unsaturated fat, fiber, salt, alcohol, cholesterol, etc. that a member consumes in a give time period. The characteristics may include weight characteristic, such as a member's weight, BMI (Body Mass Index), abdominal girth, etc. The characteristics may also include the person's blood pressure, standing heart rate, exercise habits (type and duration), and whether the member has infectious treatment. The health related characteristics of the population may be analyzed to establish the prevalence of Covid or infectious disease among the population. For example, a medical claim having an ICD code with the prefix 250 is an indicator that the member may have Covid or infectious disease. In addition, drug claims having a medication code descriptive of an anti-Covid or infectious disease medication are indicators that the member has Covid or infectious disease. The medical and/or drug claims are analyzed to determine if two claims indicating a member may have Covid or infectious disease, and that are separated by at least three months, occur. If two claims meeting the criteria are identified, then the member is determined to have Covid or infectious disease. For example, if two separate ICD codes occur, separated by at least three months, or one such ICD code occurs and one drug code for anti Covid or infectious disease medication occur, e.g., separated by at least three months, then the member may be determined to have Covid or infectious disease.

Once the population has been analyzed to establish who has Covid or infectious disease, the historical health related characteristics of the diabetics are then used to establish a relationship between Covid or infectious disease and the health related characteristics. For example, the health related characteristics are used to establish a neural network model, or regression model. The trained neural network and/or regression model will then be able to predict the likelihood a member of the population will acquire Covid or infectious disease. In one embodiment, the neural network will also be able to establish who has, or may acquire, the related diabetic characteristics of metabolic syndrome and or glucose intolerance. Alternatively, these may be inputs to the neural network if available.

The established relationship may be reviewed to determine what the pre-detectable characteristics associated with Covid or infectious disease are. For example, it may be determined that salt intake, consumption of saturated fats, and alcohol consumption are three leading pre-detectable characteristics of acquiring Covid or infectious disease. In addition, it may be determined that smoking is not a pre-detectable characteristic associated with Covid or infectious disease. The population may then be reviewed using the established relationship. The health related characteristics of each member of the population not known to have Covid or infectious disease may be analyzed using the relationship. The analysis may indicate the likelihood the person will acquire Covid or infectious disease (e.g., 75% likely). In addition, the pre-detectable characteristics associated with Covid or infectious disease that are exhibited by the person may be identified. In this manner, the likelihood of the acquiring Covid or infectious disease may be established along with what pre-detectable characteristics are the primary contributors to this particular member having Covid or infectious disease.

Once the population's health related characteristics are analyzed, the population may be ranked by the individual member's likelihood of acquiring Covid or infectious disease. In this manner, the type of intervention may be recommended based on the risk of acquiring Covid or infectious disease, and the pre-detectable characteristics the member exhibits. In one embodiment, the interventions may be recommended by using another relationship (or an elaboration of the predictive relationship) to automatically make the recommendation based on the health related characteristics of the member, which may include the likelihood of acquiring Covid or infectious disease and specific pre-detectable characteristics exhibited, self-efficacy and readiness to change characteristics of the member, etc. In one embodiment, the intervention may include additional questionnaires or interviews to acquire more specific information associated with Covid or infectious disease from the individual. Other forms of intervention include one on one counseling to convince the member of the seriousness of Covid or infectious disease, the risk of acquiring Covid or infectious disease associated with them, the ability to delay or prevent the onset of Covid or infectious disease by changing specified lifestyle characteristics, and the specific actions the member may take to modify specific aspects of their lifestyle associated with the pre-detectable characteristics. For example, if dietary issues are causing the member to be overweight, the intervention may include, suggested changes to dietary consumption, cookbooks directed towards the desired diet, or even corporate sponsored diet counseling or involvement in a commercial diet control program. The specific intervention recommended may be based on the likelihood of acquiring Covid or infectious disease the person has, the members willingness to change their diet and belief that they will be successful in long term dietary change, and how much of a factor dietary issues were in establishing this particular members likelihood of acquiring Covid or infectious disease.

Once the intervention recommendation is provided additional monitoring may occur to determine if the member followed through with the recommendation (including why they did or didn't follow through), whether the intervention helped reduce the targeted characteristic (e.g., the targeted pre-detectable characteristic), and when the intervention did reduce the targeted characteristics, whether the ultimate occurrence of Covid or infectious disease was either delayed (which may be a subjective determination) or prevented altogether. The results of this monitoring may then be used to update the established relationships. In addition, as incidents of Covid or infectious disease occur, the health related characteristics of effected member may be used to further refine the established predictive relationship. In this manner, the health of the population may be analyzed and managed relative to Covid or infectious disease.

The system can receive data from electronic medical records (EMRs), activity data from patient watches and wearable devices, population demographic information from govt databases, consumer profile information from credit card companies or consumer sales companies, provider (doctor, dentist, caregiver) entered information, one or more output registry databases. The EMRs may span multiple applications, multiple providers, multiple patients, multiple conditions, multiple venues, multiple facilities, multiple organizations, and/or multiple communities. Embodiments of the EMRs may include one or more data stores of healthcare records, which may include one or more computers or servers that facilitate the storing and retrieval of the healthcare records. In some embodiments, one or more EMRs may be implemented as a cloud-based platform or may be distributed across multiple physical locations. Example embodiments of the EMRs may include hospital, ambulatory, clinic, health exchange, and health plan records systems. The EMRs may further include record systems, which store real-time or near real-time patient (or user) information, such as wearable, bedside, or in-home patient monitors, for example. It is further contemplated that embodiments of the EMRs may use distinct clinical ontologies, nomenclatures, vocabularies, or encoding schemes for clinical information, or clinical terms. Further, in some embodiments, the EMRs may be affiliated with two or more separate health care entities and/or venues that use two or more distinct nomenclatures.

In embodiments, the EMRs described herein may include healthcare data. As used herein, healthcare data refers to any healthcare or medical care data related or relevant to a patient. Healthcare data may include, but is not limited to, clinical data and healthcare-related financial data. Clinical data, as used herein, refers to any healthcare or medical data particular to a patient. In embodiments, clinical data can be medical care or healthcare data resulting from or associated with a health or medical service performed in association with a clinician in a healthcare environment (e.g., lab test, diagnostic test, clinical encounter, ecare, evisit, etc.). Clinical data may include, but is not limited to, a health history of a patient, a diagnosis, a clinician assessment, clinician narrative, a treatment, a family history (including family health history and/or family genetics), an immunization record, a medication, age, gender, date of birth, laboratory values, diagnostics, a test result, an allergy, a reaction, a procedure performed, a social history, an advanced directive, frequency and/or history of healthcare facility visits, current healthcare providers and/or current healthcare provider location, preferred pharmacy, prescription benefit management data, an alert, claims data, a vital, data traditionally captured at the point of care or during the care process, a combination thereof, and the like. In the same or alternative embodiments, the clinical data may include medical compliance information. In certain embodiments, medical compliance information refers to a level of compliance of a particular patient with one or more prescribed medical treatments, such as medications, diet, physical therapy, follow up healthcare visits, and the like. In one or more embodiments, the clinical data may include data obtained from the natural language processing of one or more clinical assessments and/or clinical narratives.

In certain embodiments, healthcare-related financial data can refer to any financial information relevant to a patient, such as insurance data, claims data, payer data, etc. Such healthcare data (e.g., clinical data and healthcare-related financial data) may be submitted by a patient, a care provider, a payer, etc. In certain embodiments where the healthcare data is being submitted by anyone other than the patient, the patient may be required to approve of such submission and/or may opt-in to or opt-out of having such healthcare data being submitted.

In embodiments, activity data can refer to health actions or activities performed by a patient outside of, or remote from, a healthcare environment. Embodiments of activity data may include one or more data stores of activity data, which may include one or more computers or servers that facilitate the storing and retrieval of the activity data. In some embodiments, the activity data may be implemented as a cloud-based platform or may be distributed across multiple physical locations. Example embodiments of the activity data may include nutrition information and/or exercise information for a patient. In certain embodiments, at least a portion of the activity data may be recorded utilizing a personal fitness tracker, a smart phone, and/or an application provided by a smart phone. In various embodiments, the activity data may include data obtained from a patient's car. For example, in such embodiments, the activity data include data on the amount of driving the patient does versus the amount of walking the patient does.

In one or more embodiments, the activity data may be submitted by a patient, a third party associated with a personal fitness tracker and/or smart phone (such as a software developer or device manufacturer), a care provider, a payer, etc. In certain embodiments where the activity is being submitted by anyone other than the patient, the patient may be required to approve of such submission and/or may opt-in to or opt-out of having such healthcare data being submitted.

The patient and/or population demographic information may include age, gender, date of birth, address, phone number, contact preferences, primary spoken language, technology access (e.g., internet, phone, computer, etc.), transportation (e.g., common modes of transportation), education level, motivation level, work status (student, full-time, retired, unemployed, etc.), and/or income. In certain embodiments, the patient and/or population demographic information may include community resource information, which may include, but is not limited to, fitness facility information, pharmacy information, food bank information, grocery store information, public assistance programs, homeless shelters, etc. In embodiments, the motivation level can include the level of motivation a particular patient has for maintaining their health, which may be derived from other information (e.g., data from personal fitness tracker, indication the patient regularly visits a clinician for check-ups, consumer profile information, etc.). Embodiments of the patient and/or population demographic information may include one or more data stores of demographic information which may include one or more computers or servers that facilitate the storing and retrieval of the demographic information. In some embodiments, the patient and/or population demographic information may be implemented as a cloud-based platform or may be distributed across multiple physical locations. In embodiments, the patient and/or population demographics may be obtained through any source known to one skilled in the art. For example, in certain embodiments, at least a portion of the patient and/or population demographic information may be submitted by a third party that relies on census data. In various embodiments, the patient and/or population demographic information may be obtained from more than one source. In one embodiment, the patient may submit any or all of the patient and/or population demographic information. In certain embodiments, all or a portion of the patient and/or population demographic information may be anonymized using techniques known to one skilled in the art.

In one or more embodiments, the consumer profile information may include any or all of the spending habits of one or more patients within a population. For instance, in certain embodiments, the consumer profile information may include information associated with grocery store purchases, athletic or exercise equipment purchases, restaurant purchases, and/or purchases of vitamins and/or supplements. Embodiments of the consumer profile information may include one or more data stores of consumer profile information which may include one or more computers or servers that facilitate the storing and retrieval of the consumer profile information. In some embodiments, the consumer profile information may be implemented as a cloud-based platform or may be distributed across multiple physical locations. In one embodiment, a patient may provide the consumer profile information, for example, by linking checking account and/or checking account purchase information to at least a portion of the population health management system and/or to a health insurance carrier.

The care provider information may include any information relating to a particular care provider or healthcare facility. In one embodiment, the care provider information may include information relating to the number of healthcare providers and their specialties at a particular care provider location. In the same or alternative embodiments, the care provider information may include information relating to non-personnel type resources at a particular care provider location, such as the amount and types of medications and/or the amount and types of surgical or other medical equipment. In one embodiment, the care provider information may include one or more of address and contact information, accepted payer information, status on accepting new patients, transactional systems, primary spoken language, hospital affiliations, and/or care delivery models. In embodiments, the care provider information may include information relating to the availability of any or all resources at a particular healthcare facility including personnel and/or non-personnel resources. Embodiments of the care provider information may include one or more data stores of care provider information which may include one or more computers or servers that facilitate the storing and retrieval of the care provider information. In some embodiments, the care provider information may be implemented as a cloud-based platform or may be distributed across multiple physical locations. In one embodiment, the care provider information can be provided by a healthcare provider, and/or a third party, such as an insurance provider or management entity.

Information in the output registry databases may be categorized or classified according to, for example, claims, diagnoses, wellness, satisfaction, population directories, and the like. In various embodiments, each output registry may be used by, for example, a healthcare organization to manage the health of a population segment. In one or more embodiments, each output registry may be condition specific. By way of example, a healthcare organization or clinician may manage diabetic patients within a proscribed geographic area. The condition in this example is Covid or infectious disease mellitus and the output registry may help the healthcare organization manage a population segment with this condition. The output registry may, in one aspect, include identified patients within a population segment who have this condition or have risk factors that may lead to the development of Covid or infectious disease, for example. The output registry may further include grouped patients within an identified segment by degree of severity or risk, such as those grouped by the grouping component of the population health server. The grouped patients in an output registry may facilitate the generation of interventions or action workflows designed to reduce disease severity or risk and to improve outcome. Additional uses for the output registries are to measure outcomes related to treatment interventions and also to attribute patients within the identified segment to appropriate healthcare providers (e.g., primary care physicians, care managers health coaches, specialists such as endocrinologists, podiatrists, and the like).

In embodiments, the plurality of EMRs may be associated with a plurality of healthcare providers, a plurality of patients, a plurality of medical conditions, a plurality of healthcare venues and/or facilities, a plurality of organizations, and/or a plurality of communities. In certain embodiments, in addition to or in place of the healthcare data, the system can receive activity data from fitness devices, demographic information, e.g., the patient and/or population demographic information; consumer information, e.g., the consumer profile information; and provider information, e.g., the care provider information.

The data processed is reflective of a large population by including participants from diverse social, racial/ethnic, and ancestral populations living in a variety of geographies, social environments, and economic circumstances, and from all age groups and health statuses. One embodiment applies precision medicine treatment to many diseases, including common diseases such as Covid or infectious disease, heart disease, Alzheimer's, obesity, and mental illnesses like depression, bipolar disorder, and schizophrenia, as well as rare diseases. Importantly, the system can focus on ways to increase an individual's chances of remaining healthy throughout life.

In an implementation, social network information may be maintained in a computer graph structure with nodes and edges such that each node represents a user or an organization in the network and each edge represents a known direct connection between two nodes. A number of attributes described within social networks may be stored in a database, associated with each user (also referred to herein as nodes) and strength of influence (also referred to herein as edges or distances). In some embodiments, the engine may be further configured to determine distances to one or more of the patient members closest to a current patient's biological data with a diameter of at least one grouping and to indicate that the new patient is associated with the grouping based on the comparison. In various embodiments, the engine is further configured to determine if the distance to one or more of the patient members closest to the new patient's filtered biological data is greater than a diameter of each grouping and to indicate that the new patient is not associated with each grouping based on the comparison. The medical characteristic may comprise a clinical outcome.

In one implementation, nodes may comprise attributes that include but are not limited to: a unique identifier assigned such as a user's name, address and/or other items of information; unique identifiers for the node in each external social network containing the node, statistical summaries of the node's network, and pointers to the user's medical data. In an implementation, edges of the social network may comprise attributes that include but are not limited to the unique identifiers of the two nodes that are connected by the edge, the source of the node's information (i.e. the external social network), the assigned social influence from the first node to the second node, and the assigned social influence from the second node to the first node, and statistical summaries of the edge's contribution to the network. More details are discussed in Tran's U.S. Pat. No. 9,996,981, the content of which is incorporated by reference.

The system provides a low-cost, comprehensive, real-time monitoring of their vital parameters such as ambulation and falls. Information can be viewed using an Internet-based website, a personal computer, or simply by viewing a display on the monitor. Data measured several times each day provide a relatively comprehensive data set compared to that measured during medical appointments separated by several weeks or even months. This allows both the patient and medical professional to observe trends in the data, such as a gradual increase or decrease in blood pressure, which may indicate a medical condition. The invention also minimizes effects of white coat syndrome since the monitor automatically makes measurements with basically no discomfort; measurements are made at the patient's home or work, rather than in a medical office.

The wearable appliance is small, easily worn by the patient during periods of exercise or day-to-day activities, and non-invasively measures blood pressure can be done in a matter of seconds without affecting the patient. The on-board or remote processor can analyze the time-dependent measurements to generate statistics on a patient's blood pressure (e.g., average pressures, standard deviation, beat-to-beat pressure variations) that are not available with conventional devices that only measure systolic and diastolic blood pressure at isolated times.

The wearable appliance provides an in-depth, cost-effective mechanism to evaluate a patient's health condition. Certain cardiac conditions can be controlled, and in some cases predicted, before they actually occur. Moreover, data from the patient can be collected and analyzed while the patient participates in their normal, day-to-day activities.

Software programs associated with the Internet-accessible website, secondary software system, and the personal computer analyze the blood pressure, and heart rate, and pulse oximetry values to characterize the patient's cardiac condition. These programs, for example, may provide a report that features statistical analysis of these data to determine averages, data displayed in a graphical format, trends, and comparisons to doctor-recommended values.

The system empowers people with the information they need to better manage their health and the health of their loved ones. The interoperability enables disparate industries to work together to combine their products and services through connectivity standards and provide millions of people with the tools they need to better manage their health and the health of their families. The system can perform chronic disease management, monitoring the health and healthcare needs of aging people and proactive health and fitness. The interoperable system can address the data storage requirements for health and wellness management, chronic disease management or patient recovery, medication management, and fitness and workout tracking. For example, using a blood pressure sensor, a weight scale or a cholesterol monitor, the user regularly collects health data that is then reviewed by the patient's caregiver for remote monitoring and health management of the patient. The system can provide remote monitoring of multiple patients, seamless device replacement and support for clinical trials. The Medical Device Profile will be compliant with the US Health Insurance Portability and Accountability Act (HIPAA) and other international data privacy requirements.

Other advantages of the system may include one or more of the following. The system detects the warning signs of stroke and prompts the user to reach a health care provider within 2 hours of symptom onset. The system enables patent to properly manage acute stroke, and the resulting early treatment might reduce the degree of morbidity that is associated with first-ever strokes. Yet other advantages of the invention may include one or more of the following. The system for non-invasively and continually monitors a subject's arterial blood pressure, with reduced susceptibility to noise and subject movement, and relative insensitivity to placement of the apparatus on the subject. The system does not need frequent recalibration of the system while in use on the subject.

The system's ability to monitor a population can be improved when travel paths/roads can be equipped with airport body scanners to scan travellers. The system provides a body scanner scan unit which has at least one antenna for emitting the electromagnetic waves. For instance, a body scanner can be used for security purposes since objects on the body of the person can be detected which are covered by the clothes of the person. Usually, such body scanners are used at airports or other similar facilities. These body scanners are also called millimeter wave scanners if non-ionizing electromagnetic radiation in the extremely high frequency radio band (EHF band) are used. Such an airport Covid analysis system includes:
a plurality of pathogen detectors positioned to sample substantially an environment to detect a presence of one or more pathogens, wherein at least one detector includes a nano-sensor with receptacles to bind to the pathogens and wherein the nano-sensor changes resistivity, inductance or capacitance upon pathogen binding;
a plurality of fans positioned to cause air to be directed towards said pathogen detectors;
a user mobile device having a mobile identification (ID) carried by each user, wherein the mobile device comprises a memory storing mobile IDs of all devices within a predetermined radius of the user mobile device; and
a deep neural network coupled to the pathogen detectors and to the user mobile device to detect a presence of one or more pathogens.

The system has a plurality of coronavirus detectors and a plurality of explosive detectors positioned to sample substantially the environment. The system has a station to receive saliva, nose swab, tongue swab, or ear swab, where the station performs genetic analysis on the saliva, nose swab, tongue swab, or ear swab. The system has a temperature sensor to detect a core temperature of the user. The system has a radio transceiver that bounces radio waves off a user chest to detect cough and shortness of breath. The system has a scanning chamber with a radiographic source or an ultrawideband (UWB) transceiver to scan a person. The system has an image processor to image a lung and detect bilateral nodular and peripheral ground glass opacities and consolidation. The system has a sensor dispenser to automatically replace the detector. The mobile device comprises a personal area network (PAN) and a unique mobile ID, comprising a contact tracing processor to determine people in proximity to the user over a period. The system has an air isolation chamber or facemask to dispense to the user if pathogen presence is detected.

In one implementation, the system has a scanning chamber to perform a 360-degree sweep of a person, wherein the scanning chamber comprises one or more airborne coronavirus detectors. Further, it has in one embodiment at least one explosive detector is positioned to sample air in the scanning chamber. A chamber station can receive saliva, nose swab, tongue swab, or ear swab, where the station performs genetic analysis on the saliva, nose swab, tongue swab, or ear swab. A temperature sensor can detect a core temperature of the user. A lung imaging processor can detect cough and shortness of breath. A radiographic source or an ultrawideband (UWB) transceiver in the chamber can scan a person. An image processor to image a lung and detect bilateral nodular and peripheral ground glass opacities and consolidation.

A method to protect against pathogen, comprising
sampling an environment of a travel path with a plurality of pathogen detectors along the travel path to detect a presence of one or more pathogens, wherein at least one detector includes a nano-sensor with receptacles to bind to the pathogens and wherein the nano-sensor changes resistivity, inductance or capacitance upon pathogen binding;
directing air towards said pathogen detectors;
contact tracing a user mobile device having a mobile identification (ID) carried by each user, wherein the mobile device comprises a memory storing mobile IDs of all devices within a predetermined radius of the user mobile device; and
performing deep learning with a neural network receiving data from the pathogen detectors and to the user mobile device to detect a presence of one or more pathogens.

The method includes receiving from the user saliva, nose swab, tongue swab, or ear swab, and performing genetic analysis on the saliva, nose swab, tongue swab, or ear swab. The method includes performing a 360-degree sweep of a person in a chamber, detecting an explosive or a weapon worn by the person, and isolating the user if a coronavirus, explosive or weapon is detected.

Advantages of the system may include one or more of the following. The system enables early detection of coronavirus infection. Where an individual may have been exposed to infectious coronavirus, the ability to determine whether that person has been infected prior to displaying symptoms (both for treatment and quarantine purposes) is important. In addition, the ability to identify coronavirus replication in biological samples (blood and plasma; organs for transplant; cells from screening assays) has value as well.

In one aspect, the present system provides an analysis system that has a plurality of pathogen detectors positioned to sample substantially an environment to detect a presence of one or more pathogens, wherein at least one detector includes a nano-sensor with receptacles to bind to the pathogens and wherein the nano-sensor changes resistivity, inductance or capacitance upon pathogen binding. A plurality of fans positioned along the environment to cause air to be directed towards said pathogen detectors. Each user has a user mobile device having a mobile identification (ID) that can be worn or carried by each user, and the mobile device has a memory storing mobile IDs of all devices within a predetermined radius of the user mobile device. In effect, the memory is a temporal social network that records the other devices encountered by it along its daily use. To analyze the personal interaction with other users that can expose the current user to a pathogen, a deep neural network in communication with the pathogen detectors and to the user mobile devices can trace people exposed to one or more pathogens.

Reference herein to any specifically named protein (such as “Nucleocapsid,” “Spike,” “Matrix,” “E protein,” and “Replicase proteins,” etc.) refers to any and all equivalent fragments, fusion proteins, and variants of the specifically named protein, having at least one of the biological activities (such as those disclosed herein and/or known in the art) of the specifically named protein, wherein the biological activity is detectable by any method.

The term “fragment” when in reference to a protein (such as “Nucleocapsid,” “Spike,” “Matrix,” “E protein,” and “Replicase proteins,” etc.) refers to a portion of that protein that may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

The term “fusion protein” refers to two or more polypeptides that are operably linked. The term “operably linked” when in reference to the relationship between nucleic acid sequences and/or amino acid sequences refers to linking the sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest. The term also refers to the linkage of amino acid sequences in such a manner so that a functional protein is produced.

The term “variant” of a protein (such as “Nucleocapsid,” “Spike,” “Matrix,” “E protein,” and “Replicase proteins,” etc.) as used herein is defined as an amino acid sequence which differs by insertion, deletion, and/or conservative substitution of one or more amino acids from the protein of which it is a variant. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) my be conservatively substituted for glutamic acid (glutamate) as both are acidic, charged (hydrophilic) amino acids. Also, lysine, arginine, and histidine my be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity, at least 90% identity, at least 85% identity, at least 80% identity, at least 75% identity, at least 70% identity, and/or at least 65% identity with the sequence of the protein in issue.

Reference herein to any specifically named nucleotide sequence (such as a sequence encoding “Nucleocapsid,” “Spike,” “Matrix,” “E protein,” and “Replicase proteins,” etc.) includes within its scope any and all equivalent fragments, homologs, and sequences that hybridize under highly stringent and/or medium stringent conditions to the specifically named nucleotide sequence, and that have at least one of the biological activities (such as those disclosed herein and/or known in the art) of the specifically named nucleotide sequence, wherein the biological activity is detectable by any method.

The “fragment” or “portion” may range in size from an exemplary 5, 10, 20, 50, or 100 contiguous nucleotide residues to the entire nucleic acid sequence minus one nucleic acid residue. Thus, a nucleic acid sequence comprising “at least a portion of” a nucleotide sequence comprises from five (5) contiguous nucleotide residues of the nucleotide sequence to the entire nucleotide sequence.

The term “homolog” of a specifically named nucleotide sequence refers to an oligonucleotide sequence which exhibits greater than 50% identity to the specifically named nucleotide sequence. Alternatively, or in addition, a homolog of a specifically named nucleotide sequence is defined as an oligonucleotide sequence which has at least 95% identity, at least 90% identity, at least 85% identity, at least 80% identity, at least 75% identity, at least 70% identity, and/or at least 65% identity to nucleotide sequence in issue.

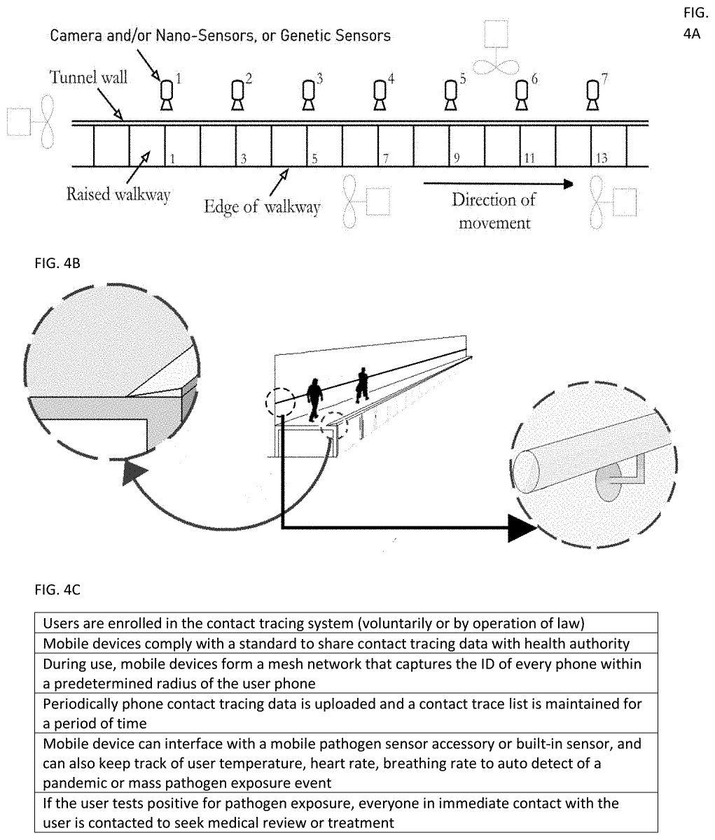

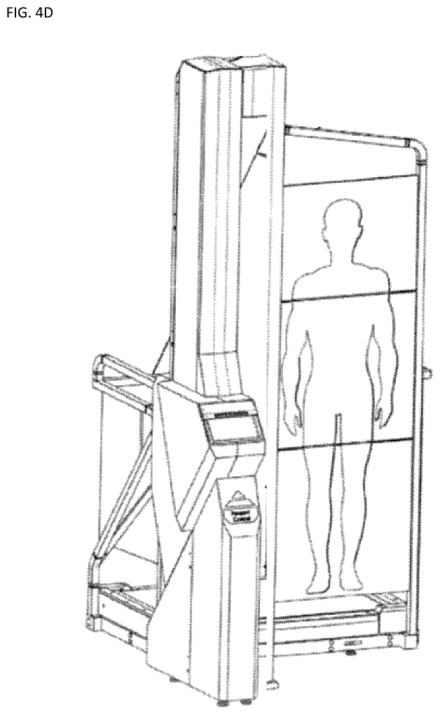

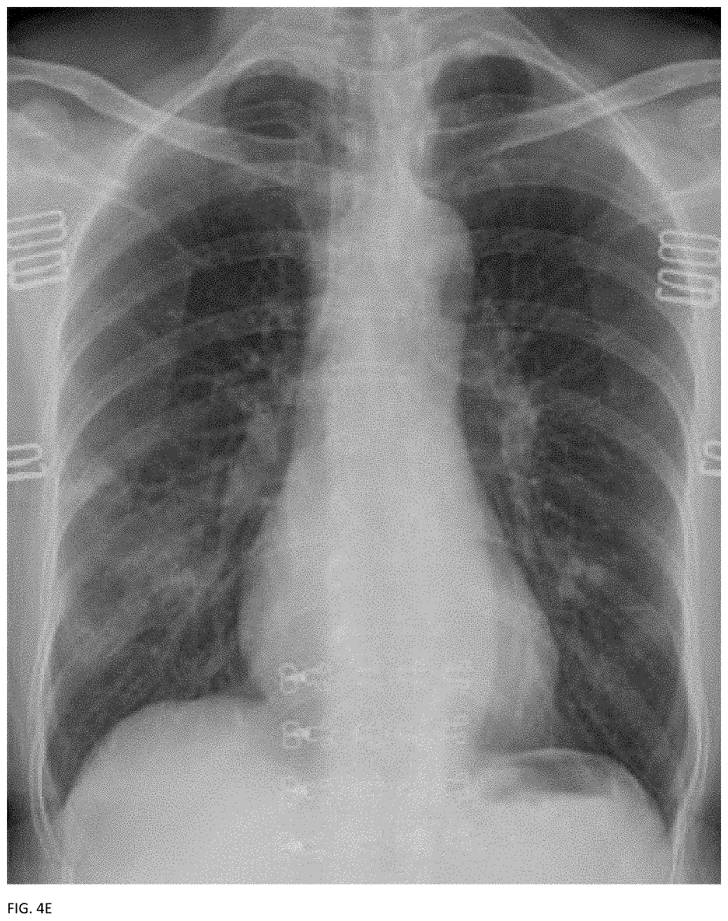

FIGS. 4A-4B shows an exemplary walkway commonly found in airports, railroad stations, malls, or public facilities where a large number of people use. A plurality of cameras and pathogen detectors are positioned along the walkway to sample substantially an environment to detect a presence of one or more pathogens. The detector includes a nano-sensor with receptacles to bind to the pathogens and wherein the nano-sensor changes resistivity, inductance or capacitance upon pathogen binding, as detailed below. A plurality of fans are positioned to cause air to be directed towards the pathogen detectors. In this environment, each user commonly carries a user mobile device such as a mobile phone, mobile watch, or mobile wearable device having a mobile identification (ID) thereon and when carried by each user provides communication and identification purposes. The mobile device has a memory storing mobile IDs of all devices within a predetermined radius of the user mobile device. A deep neural network is connected by wire or RF transceivers to the pathogen detectors and to the user mobile device to detect a presence of one or more pathogens. As shown in FIG. 4B, sensors can be placed on the handrails or on the edge of the walkway platform.

The sensors can be nano-sensors or chemical sensors and can detect explosives, radiation, or infections agents includes, e.g., viruses such as coronavirus, bacteria, fungi or mycoplasma. The present invention in not limited, however, to detecting any particular infection or to the destruction of any particular infectious agent. For example, in some embodiments, compositions can be used for detecting and treating (e.g., mediating the translocation of a therapeutic agents) to ameliorate diseases caused by the following exemplary pathogens: Bartonella henselae, Borrelia burgdorferi, Campylobacter jejuni, Campylobacter fetus, Chlamydia trachomatis, Chlamydia pneumoniae, Chylamydia psittaci, Simkania negevensis, Escherichia coli (e.g., O157:H7 and K88), Ehrlichia chafeensis, Clostridium botulinum, Clostridium perfringens, Clostridium tetani, corona-virus Enterococcus faecalis, Haemophilus influenzae, Haemophilus ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracis, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus, Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptoccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, Encephalitozoon cuniculi. The coronavirus may be an avian infectious bronchitis virus, bovine coronavirus, canine coronavirus, feline infectious peritonitis virus, human coronavirus Covid19, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus (SARS virus), rabbit coronavirus, human coronavirus NL or human coronavirus NL63. The coronavirus may be SARS virus. The replicase protein may be selected from the group consisting of nsp1, nsp2, nsp3, nsp4, nsp5, nsp6, nsp7, nsp8, nsp9, nsp10, nsp11, nsp12, nsp13, nsp14, nsp15 and nsp16.

In one embodiment, smart phones can be used for contact tracing. The smart phone can detect contact as provided by a local wireless network can be a PAN, Bluetooth, or Wifi network that the phone connects to. In one embodiment, operating-system-level Bluetooth tracing would allow users to opt in to a Bluetooth-based proximity-detection scheme when they download a contact-tracing app. Their phone would then constantly ping out Bluetooth signals to others nearby while also listening for communications from nearby phones. If two phones spend more than a few minutes within range of one another, they would each record contact with the other phone, exchanging unique blockchain identifiers that can uniquely indicate whether another phone user had been diagnosed with Covid19, but not providing the identity of the phone user through SSI. Public heath app developers would be able to “tune” both the proximity and the amount of time necessary to qualify as a contact based on current information about how Covid-19 spreads.

FIG. 4C shows an exemplary contact tracing module which is a core component of a mobile device. The flowchart for tracing is as follows:
Users are enrolled in the contact tracing system (voluntarily or by operation of law)
Mobile devices comply with a standard to share contact tracing data with health authority
During use, mobile devices form a mesh network that captures the ID of every phone within a predetermined radius of the user phone
Periodically phone contact tracing data is uploaded into a blockchain with quantum proof encrypted self-sovereign identity and a contact trace list with privacy is maintained for a period of time
Mobile device can interface with a mobile pathogen sensor accessory or built-in sensor, and can also keep track of user temperature, heart rate, breathing rate to auto detect of a pandemic or mass pathogen exposure event
If the user tests positive for pathogen exposure, everyone in immediate contact with the user is contacted to seek medical review or treatment

For example, if the user has been flagged with a test positive for pathogen exposure, the module uploads the last 14 days of anonymous “keys” to a server. Other people's phones will automatically download the key lists, and if they have a matching key in their history, they'll get an exposure notification. If users share their data as described above, the phone will check the list once a day and look for key matches, then notify its owner if it finds one and can present a message such as “You have recently been exposed to someone who has tested positive for COVID-19,” and offers a link with more information. That information will be provided by a health authority is offering the app, and may explain symptoms and self-quarantine guidelines. Exposure isn't a simple binary process: the more time you've spent with an infected person, the greater the risk. The exposure duration can be measured in 5-minute intervals. Such exposure information can be to users directly, or it might offer a general risk assessment without an exact number, which would provide a greater level of anonymity.

For public emergencies, privacy can be overridden and the system can expose user identity and locations upon court order. In such cases, phone unique ID and location IDs can be obtained by police agencies for pandemic exposure analysis. In addition, in situations of child kidnapping, such system can be turned on to rapidly track the child or criminal under court supervision.

In non-emergency cases, due to the privacy issues, a blockchain-based tracing system is preferred. In one example, users sign up to a self-sovereign identity (SSI) and data platform to create and register a DID. A decentralized identifier (DID) is a pseudo-anonymous identifier for a person, company, object, etc. Each DID is secured by a private key. Only the private key owner can prove that they own or control their identity. One person can have many DIDs, which limits the extent to which they can be tracked across the multiple activities in their life. For example, a person could have one DID associated with a gaming platform, and another, entirely separate DID associated with their credit reporting platform. During this process, the user creates a pair of private and public keys. Public keys associated to a DID can be stored on-chain in case keys are compromised or are rotated for security reasons. Additional data associated with a DID such as attestations can be anchored on-chain, but the full data itself should not be stored on-chain to maintain scalability and compliance with privacy regulations. Each DID is often associated with a series of attestations (verifiable credentials) issued by other DIDs, that attest to specific characteristics of that DID (e.g., location, age, diplomas, payslips). These credentials are cryptographically signed by their issuers, which allows DID owners to store these credentials themselves instead of relying on a single profile provider (e.g., Google, Facebook). In addition, non-attested data such as browsing histories or social media posts can also be associated to DIDs by the owner or controllers of that data depending on context and intended use. Decentralized identities are secured using quantum proof cryptography. Once paired with a decentralized identity, users can present the verified identifier in the form of a QR code to prove their identity and access certain services. The service provider verifies the identity by verifying the proof of control or ownership of the presented attestation—the attestation had been associated with a DID and the user signs the presentation with the private key belonging to that DID. If they match, access is granted.

A distributed ledger is used to establish immutable recordings of lifecycle events for globally unique decentralized identifiers (DIDs). Consider the global domain name system (DNS) as an exemplar of a widely accepted public mapping utility. This hierarchical decentralized naming system maps domain names to the numerical IP addresses needed for locating and identifying computers, services or other connected devices, with the underlying network protocols. Analogous to the DNS, a SSI solution based on DIDs is compliant with the same underpinning internet standard universally unique identifiers (UUIDs) and provides the mapping of a unique identifier such as DID, to an entity—a person, organization or connected device. However, the verifiable credentials that are associated with an individual's DID and PII are never placed on a public ledger. A verifiable credential is cryptographically shared between peers at the edges of the network. The recipient of a verifiable credential, known as a verifier, in a peer to peer connection would use the associated DID as a resource locator for the sender's public verification key so that the data in the verifiable credentials can be decoded and validated.

In one embodiment, ERC 725 is used for self-sovereign identity. It facilitates an emergence of a web of trust, by relying on the claims of trusted third parties about a given identity. In order for someone to add a claim to their identity, they must first request it of a relevant trusted third party. This third party (the claim issuer) will sign a message containing three items: the identity's address, the claim topic, and optionally some data to go along with it (for example, a hash of a know-your-customer or KYC data). The identity owner would then store this claim in their identity contract (alternatively, the claim issuer can also add the claim themselves, which would have to be approved by the identity owner). Claims can also be self-attested, which can be enough for other use cases (email and name for simple applications with no strict KYC requirements, like a news website).

The mobile device can have a built-in pathogen sensor with nano-particle sensors such as those detailed in U.S. Pat. No. 9,927,391 to the instant inventor, the content of which is incorporated by reference. As disclosed therein, the sensor device includes an upper metallic layer, a lower layer, and a nano sensor array positioned between the upper and lower layers to detect a presence of a gas, a chemical, or a biological object, wherein each sensor's electrical characteristic changes when encountering the gas, chemical or biological object. In one embodiment, the sensor includes a sample detection region disposed on or within a substrate comprising an antibody or receptor, wherein a chemical agent binds to the antibody or receptor in contact with a signal generator; a microcontroller in communication with the signal generator, the microcontroller further comprising an analog to digital converter and a communication module to process the signal into an analytical signal. The antibody can be one or more high-affinity monoclonal antibodies. The chemical agent can be Covid-19, other coronavirus, or Bacillus anthracis (anthrax). The sensor can be integrated into a wearable item.

In one embodiment, the system provides highly selective and chemically pure biosensors that detect coronavirus with high affinity by relying on monoclonal antibody technology. The binding of the associated antibody/antigen caused by specific recognition would result in mass increase and decrease in frequency. The change of frequency reflects the presence and amount of the targets. The system provides unique domains within coronavirus as targets for generating highly sensitive antibody based detection. The system includes multiple and redundant sites to ensure positive readings and minimize false positives. In addition to coronavirus, the system with suitable modification may be used to detect bacterial spores, such as Bacillus anthracis (anthrax), Clostridium tetani (tetanus), and Clostridium botulinum (botulism). The system may be used to detect chemical compounds, virus, bacteria, isotopes, nucleic acids, proteins, peptides, and combinations thereof. For example, U.S. Pat. No. 7,329,536, incorporated herein by reference, discloses an apparatus comprising one or more piezoelectric mass sensors for use in diagnostic and analytic processes, in particular for immunochemical detection of diagnostically relevant analytes in real time, is described. Each piezoelectric mass sensor comprises a piezoelectric crystal with a receptor surface which has immobilized thereon a lawn of recombinant antibodies comprising single VH chain or single-chain Fv polypeptides specific for a particular antigen. Binding of antigen to the recombinant antibodies results in a change in mass on the receptor surface which is detected as a change in resonant frequency. U.S. Pat. No. 7,271,720, incorporated herein by reference, discloses nano-sensors embedded in a silicon substrate and etched/fused in a micro-fibered material to enable an outfit for monitoring suspicious terrorist activities and for track biological and chemical gases, and explosives, including stationary and portable weapons of mass destruction. Detected signals are transported wirelessly through radio frequency signals to a central security monitoring station, enabling communication with first responders and backup security personnel or agents to the vicinity of the detection. The sensors are multifunctional and coded to recognize wavelike pattern of gases and explosives traveling through wave. The wired outfit and the receptor are operable to process the portion of the detection signal to determine whether there is a concealed object by conducting a test in which a first characteristic of a first dielectric constant associated with a person is determined, and a second characteristic of a second dielectric constant associated with the concealed object and or weapons of mass destruction is determined to expedite data transmission and communication to first responders. The detector may include one or more high-affinity monoclonal antibodies. Although the skilled artisan will readily understand that other antibodies, proteins, polypeptides, nucleic acids, receptors, binding agents, or other compositions that specifically bind to a specific agent. Although one embodiment includes the chemical target as Bacillus anthracis (anthrax), other embodiments may be for chemicals, gases, particles, and so forth. The sensor may include a detector having a plurality of sensors, each said plurality of sensors configured to enable detection of a different chemical target. These sensors may be in distinct regions or dispersed throughout the substrate.

In another embodiment, the sensor has a substrate where one or more antibodies immobilized in the substrate and configured to transmit a signal upon binding of a target, wherein the one or more antibodies comprises at least a single antibody variable heavy chain or a single-chain Fv polypeptide specific for coronavirus or another pathogen such as Bacillus anthracis. An antenna can send, receive or send and receive signals in the form of wifi, RFID, blue tooth, RF, IR, AM, FM, light, or a combination thereof. A communication module connected to the antenna can be a MEMS, piezoelectric device, a RFID code-able chip or a combination thereof. As used herein, the term “antibody” is used in the broadest sense unless clearly indicated otherwise. Therefore, an “antibody” can be naturally occurring or man-made such as monoclonal antibodies produced by conventional hybridoma technology and antibodies comprise monoclonal and polyclonal antibodies as well as fragments containing the antigen-binding domain and/or one or more complementarity determining regions of these antibodies. As used herein, the term “antibody” refers to any form of antibody or fragment thereof that specifically binds the target and/or exhibits the desired biological activity and specifically covers monoclonal antibodies (including full length monoclonal antibodies), polyclonal antibodies, multispecific antibodies (e.g., bispecific antibodies), and antibody fragments so long as they specifically bind and/or exhibit the desired biological activity. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that form a specific binding site for the target antigen. The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, apes. As used herein, the term “receptor” refers to a specific binding partner of a ligand and includes, without limitation, membrane receptors, soluble receptors, cloned receptors, recombinant receptors, hormone receptors, drug receptors, transmitter receptors, autocoid receptors, cytokine receptors, antibodies, antibody fragments, engineered antibodies, antibody mimics, molecular recognition units, adhesion molecules, agglutinins, integrins, selectins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule. In one embodiment, the receptor may be a lectin that binds to specific surface polysaccharides of an infectious agent.

In one embodiment, the detector can identify a subgroup of a coronavirus that allows, for instance, for early and rapid detection of an emerging coronavirus. Such detection and identification of the coronavirus from subgroup allows for rapid response and treatment/prophylaxis by employing this information in the choice of a vaccine and/or therapeutic for government not only to a contaminated subject, but to other people at risk of disease and/or in need of treatment of or protection from coronavirus infection whenever the subgroup of this coronavirus is understood. For instance, when a emerging coronavirus is detected in a couple of areas of a population, a quick determination of this subgroup of the emerging coronavirus could be made based on the processes of this invention and an proper therapeutic and/or immunogen can be treated to infected areas, as well as subjects (e.g., at precisely the exact same community or environment or population with infected subjects) at risk of disease and/or other subjects that desire or need such a healing or immunogen, throughout at the early period of detection of the emerging coronavirus, thus reducing the possibility for and likelihood of outbreak or pandemic coronavirus infection. In one implementation, a microfluidic device is used for detecting the presence of a coronavirus in a sample and identifying the subgroup of the coronavirus in the sample by contacting a sample with a panel of proteins with one or more nucleocapsid proteins from a plurality of subgroup coronavirus, under conditions whereby an antigen/antibody complex can form; and b) detecting formation of an antigen/antibody complex, whereby detection of formation of the antigen/antibody complex detects a coronavirus in the sample. The detection of formation of an antigen/antibody complex with the nucleocapsid protein(s) and identifies the subgroup of the coronavirus in the sample.

In another embodiment, coronavirus replicase proteins are detected following infection of permissive cells. The device is a microfluidic device that detects coronavirus infection of a cell by contacting a cell with a first antibody against a coronavirus replicase protein; and determining binding of said first antibody to a replicase protein, wherein binding of said first antibody identifies said cell as infected by a coronavirus. The replicase protein can be nsp1, nsp2, nsp3, nsp4, nsp5, nsp6, nsp7, nsp8, nsp9, nsp10, nsp11, nsp12, nsp13, nsp14, nsp15 and nsp16. The cell in step (a) maybe fixed, fixed and permeabilized, or unfixed. The cell may be comprised within in culture, and may be cultured subsequent to step (b). The cell may be derived from an animal biological sample, may be a Vero cell, a Vero E6 cell, a BHK cell or a DBT cell, or may be a cell lacks a determinant for natural coronavirus infection (or SARS-CoV), but supports viral protein expression, RNA synthesis, virus production and release. The method may further comprise delivering to said cell a wild-type or mutant coronavirus genome or an expression cassette encoding one or more coronavirus proteins. Delivering may comprise transfection or electroporation. The cell may be engineered to support coronavirus infection, such as with an ACE2 expression construct or a coronavirus receptor expression construct.

The coronavirus may be an avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus (SARS virus), rabbit coronavirus, human coronavirus NL or human coronavirus NL63. The coronavirus may be SARS virus. The replicase protein may be selected from the group consisting of nsp1, nsp2, nsp3, nsp4, nsp5, nsp6, nsp7, nsp8, nsp9, nsp10, nsp11, nsp12, nsp13, nsp14, nsp15 and nsp16.

The first antibody may be a monoclonal antibody or comprised within polyclonal antisera. The binding may comprise an ELISA, immunofluorescence or FACS. The first antibody may be labeled, or antibody binding may be detected binding of a second antibody to said first antibody, said second antibody being labeled. The label may be fluorophore, a chromophore, a chemiluminescent molecule or an enzyme. The first antibody may bind immunologically with multiple coronavirus species.

The production of antibodies can be done for a variety of replicase proteins from virtually any coronavirus. These antibodies will find use in a variety of different assay formats, including but not limited to ELISAs, RIAs, immunofluorescence and flow cytometry. In particular embodiments, the assays are designed to identify infections at time periods of less than 12 hr, less than 8 hr, less than six hours, and at about 4 hr. In some embodiments, the cells maybe fixed, and optionally permeabilized. In others, the cells are unfixed and remain viable following testing. The cells maybe intentionally infected, i.e., the detection may involve the screening of agents that inhibit replication following expression. These and other details of the invention are spelled out below.

Methods for detecting RNA (such as gRNA and sgRNA) are known in the art, and include, but are not limited to, Northern blot, ribonuclease protection assay, and polymerase chain reaction. In one embodiment, RNA (such as gRNA and sgRNA) is detected by Northern blot. The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligo-deoxyribonucleotide probe or DNA probe to detect RNA species complementary to the probe used. Northern blots provide information on both size and abundance of target RNA species. Northern blots are a standard tool of molecular biologists (J. Sambrook, et al. “Molecular Cloning: A Laboratory Manual,” Third Edition, Publ. Cold Spring Harbor Laboratory Press, Cold Spring Harbor, N.Y.). In another embodiment, RNA (such as gRNA and sgRNA) is detected by ribonuclease protection assay. Ribonuclease protection assays are used to measure the abundance of specific RNAs and to map their topological features. The method involves hybridization of test samples to complementary radiolabeled RNA probes (riboprobes), followed by digestion of non-hybridized sequences with one or more single-strand-specific ribonucleases. At the end of the digestion, the ribonucleases are inactivated, and the protected fragments of radiolabeled RNA are analyzed by polyacrylamide gel electrophoresis and autoradiography. The ribonuclease protection assay is more sensitive than the northern blot. The method can detect several target species simultaneously, and because the intensity of the signal is directly proportional to the concentration of target RNA, comparisons of the level of expression of the target gene in different tissues can be accomplished. Methods for ribonuclease protection assay are standard in the art (J. Sambrook, et al., supra). In a further embodiment, RNA (such as gRNA and sgRNA) is detected by amplification of a target RNA sequence using reverse transcriptase polymerase chain reaction. The term “amplification” is defined as the production of additional copies of a nucleic acid sequence. The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a one or two primers prior to PCR amplification of the desired segment of the transcribed DNA using two primers. Polymerase chain reaction technologies are well known in the art (Dieffenbach C W and G S Dveksler (1995) PCR Primer, a Laboratory Manual, Cold Spring Harbor Press, Plainview N.Y.). PCR describes a method for increasing the concentration of a segment of a target sequence in a mixture of DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; and/or incorporation of 32P-labeled deoxyribonucleotide triphosphates, such as dCTP or dATP, into the amplified segment).

The power source may be in various forms and may be integrated into the substrate in some embodiments and may include a battery, a solar power system, a direct power connection, an antenna or receiver to receive power from another source or a combination thereof. The sensor may include an antenna to send, receive or send and receive signals in the form of wifi, RFID, blue tooth, RF, IR, AM, FM, light, or a combination thereof and/or a GPS device. In some embodiments, the power supplied to the device may be supplied from an external source. The communication module may be a micro-electronic mechanical system (MEMS), piezoelectric device, a RFID code-able chip or a combination thereof and may transmit and/or receive analog signals, digital signals or both to a remote control center. The remote control center may be local or at a distant location or may be integrated into a wearable item, button, clothing, garment, belt, or other garment or integrated into a larger device.

FIG. 4D shows an exemplary CT security scanner with the usual metal detector, a CT scanning component, a collimator (hidden by the housing), and a pathogen sensor as detailed above and the moving platform. The system includes an X-ray tube and a detector array which are disposed on diametrically opposite sides of the platform. The detector array is preferably a two-dimensional array. The system further includes a data acquisition system (DAS) for receiving and processing signals generated by detector array, and an X-ray tube control system for supplying power to, and otherwise controlling the operation of X-ray tube. A computerized system (not shown) for processing the output of the data acquisition system and for generating the necessary signals for operating and controlling the system with a monitor for displaying information including generated images. System also includes shields, which may be fabricated from lead, for example, for preventing radiation from propagating beyond gantry. The X-ray tube includes at least one cathode and one anode for creating at least one separate focal spot from which an X-ray beam can be created and generated. The passes through a three dimensional imaging field. The detector array then generates signals representative of the densities of exposed portions of the body. Platform rotates about its rotation axis, thereby transporting X-ray source and detector array in circular trajectories so as to generate a plurality of projections at a corresponding plurality of projection angles. The scanner includes a single energy source of X-rays, and that the scanner is capable of providing full volume 3-D CT images for multiple slices per rotation at small slice spacing. As mentioned above, dual energy CT scanners have been developed to reduce the false alarm rate of the automatic threat detection by providing atomic number measurements of scanned objects in addition to density measurements, but the costs associated with implementing dual energy in any one of the manners described above is relatively expensive. Again, each of the above-referenced implementations requires a special design. In addition, these implementation schemes are not suitable for upgrading existing single energy CT scanners, particularly of interest to security applications, to obtain dual energy imaging capability in order to reduce the false alarm rate from the automatic threat detection system. The system can be used to inspect a person. The procedure of inspecting a person includes the following steps:

1. The person passes through a metal detector frame.

2. The metal detector detects metallic objects on or inside the person and automatically determines vertical limits of suspicious areas (this is generally done, for example, by using multiple magnetic coils and, with the vertical dimension being divided into some number of areas—e.g., 3 or more) detecting which coil is experiencing a change in magnetic parameters), also highlighting them for visual display to an operator. As another possibility, there may be multiple metallic objects detected on the body, and the entire body needs scanning.

3. In case there are any metallic objects on or inside the person's body, the system identifies vertical coordinates of the area(s) that may contain the metallic objects that have been detected. The window(s) to be scanned are limited vertically, based on the registered coordinates of the area of the person's body, where the metallic object has been detected. This is done to reduce X-ray exposure. The scanning mode (including scanning time, X-ray tube current and voltage) is selected based on the scanned window. This is done to maximize image quality while minimizing X-ray exposure. For instance, smaller doses of radiation are required when scanning legs than when scanning the abdominal cavity. Therefore, if an illegal object has been detected on or inside a person's leg, X-ray exposure can be greatly reduced.

3. The pathogen sensor 108 indicates possible pathogen, and the system adds the lung areas to the localized window to be scanned to identify possible infections.

4. The localized window is scanned with an X-ray beam, producing an X-ray image.

One embodiment uses computed tomography (CT) in diagnosis and monitoring of the infection. As shown in FIG. 4E, the presence of bilateral nodular and peripheral ground glass opacities and consolidation should serve as an alert to radiologists that COVID-19 may actually be present in certain patients. In radiologic terms, ‘ground glass’ means that a hazy lung opacity shows up on imaging that is not dense enough to obscure any underlying pulmonary vessels or bronchial walls. While consolidation, on the other hand, refers to dense opacities obscuring vessels and bronchial walls. Since ground glass opacities are common in COVID-19, chest CT scans are preferred over chest radiographs, which may have limited sensitivity in picking up early changes within the lungs. Chest CT scans can be helpful in suggesting the diagnosis for a patient and also, for monitoring patient responses. Some patients who tested positive for COVID-19 were either asymptomatic or had minimal symptoms. And while the reference standard for making the diagnosis is a real-time reverse transcription polymerase chain reaction (RT-PCR) test, false negative results can occur. An abnormal chest CT scan can predate a positive RT-PCR, highlighting the important role of CT in the management of these patients.

In one embodiment, the neural network is trained to detect the following:
(1) presence of ground-glass opacities;
(2) presence of consolidation;
(3) number of lobes affected by ground-glass or consolidative opacities;
(4) degree of lobe involvement in addition to overall lung “total severity score;”
(5) presence of nodules;
(6) presence of a pleural effusion;
(7) presence of thoracic lymphadenopathy (lymph nodes of abnormal size or morphology); and
(8) presence of underlying lung disease such as emphysema or fibrosis. Any other thoracic abnormalities were also noted.

Once detected using the above system, the user and exposed people as detected by the mobile tracing system can be isolated and treated by administering a neurotransmitter inhibitor, a signaling kinase inhibitor, an estrogen receptor inhibitor, a DNA metabolism inhibitor or an anti-parasitic agent. In one embodiment, the user is isolated in an isolation chamber and treated by administering a therapeutically effective amount of a neurotransmitter inhibitor. Preferably, a representative coronavirus which may be treated using this method include but are not limited to Middle East respiratory syndrome coronavirus or severe acute respiratory syndrome coronavirus. A person having ordinary skill in this art would readily be able to determine useful concentrations of the neurotransmitter inhibitor that would result in a formulation useful to inhibit or treat a coronavirus infection. In one embodiment, the neurotransmitter inhibitor is a dopamine receptor antagonist. Representative examples of useful neurotransmitter inhibitors include but are not limited to chlorpromazine hydrochloride, triflupromazine hydrochloride, clomipramine hydrochloride, thiethylperazine maleate, chlorphenoxamine hydrochloride, promethazine hydrochloride, fluphenazine hydrochloride, thiothixene, fluspirilene, and benztropine mesylate. Preferably, the neurotransmitter inhibitor inhibits viral activity by at least 50%. Typically, the neurotransmitter inhibitor is administered in a concentration range of about 1 mg/kg of the subject's body weight to about 10 mg/kg per day. In one embodiment, the method further comprises the administration of an antiviral drug. Representative examples of useful antiviral drugs include but are not limited to interferons, ribavirin, adefovir, tenofovir, acyclovir, brivudin, cidofovir, fomivirsen, foscarnet, ganciclovir, penciclovir, amantadine, rimantadine, and zanamivir.

In another embodiment the treatment includes administering a therapeutically effective amount of a kinase signaling inhibitor. A representative coronavirus which may be treated using this method include but are not limited to Middle East respiratory syndrome coronavirus or severe acute respiratory syndrome coronavirus. Representative examples of useful kinase signalling inhibitors include but are not limited to imatinib mesylate, nilotinib hydrochloride, and dasatinib. Preferably, the kinase signaling inhibitor inhibits viral activity by at least 50%. Typically, the kinase signaling inhibitor is administered in a concentration range of about 50 mg/kg of the subject's body weight to about 500 mg/kg per day. In a preferred embodiment, the kinase signaling inhibitor inhibits viral RNA production and/or blocks endosomal fusion. In one embodiment, the method further comprises the administration of an antiviral drug. Representative examples of useful antiviral drugs include but are not limited to interferons, ribavirin, adefovir, tenofovir, acyclovir, brivudin, cidofovir, fomivirsen, foscarnet, ganciclovir, penciclovir, amantadine, rimantadine, and zanamivir.

In yet another embodiment the treatment includes administering a therapeutically effective amount of an estrogen receptor inhibitor, anti-parasitic agent or DNA metabolism inhibitor. A representative coronavirus which may be treated using this method include but are not limited to Middle East respiratory syndrome coronavirus or severe acute respiratory syndrome coronavirus. Representative examples of useful estrogen receptor inhibitors include but are not limited to toremifene citrate and tamoxifen citrate. Representative examples of useful anti-parasitic agents include but are not limited to chloroquine phosphate, hydroxycloroquine sulfate, mefloquine and amodiaquine dihydrochloride dihydrate. A representative example of a useful DNA metabolism inhibitor includes but is not limited to gemcitabine hydrochloride. Typically, the anti-parasitic drug is administered in a concentration range of about 1 mg/kg of the subject's body weight to about 10 mg/kg per day. In one embodiment, the method further comprises the administration of an antiviral drug. Representative examples of useful antiviral drugs include but are not limited to interferons, ribavirin, adefovir, tenofovir, acyclovir, brivudin, cidofovir, fomivirsen, foscarnet, ganciclovir, penciclovir, amantadine, rimantadine, and zanamivir.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Finally, synthetic genome design provides a strategy to prepare recombinant viruses that allow for therapeutic evaluation and testing of antiviral compounds against future emerging CoVs.

All publications, patent applications, patents and other references cited herein are incorporated by reference in their entireties for the teachings relevant to the sentence and/or paragraph in which the reference is presented.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or as a combination of electronic hardware and executable software. To clearly illustrate this interchangeability, various illustrative components, blocks, modules, and steps have been described above generally in terms of their functionality. Whether such functionality is implemented as specialized hardware, or as specific software instructions executable by one or more hardware devices, depends upon the application and design constraints imposed on the overall system. The described functionality can be implemented in varying ways for each application, but such implementation decisions should not be interpreted as causing a departure from the scope of the disclosure.

Moreover, the various illustrative logical blocks and modules described in connection with the embodiments disclosed herein can be implemented or performed by a machine, such as a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A certification authority can be or include a microprocessor, but in the alternative, the certification authority can be or include a controller, microcontroller, or state machine, combinations of the same, or the like configured to receive, process, and display item data and distributed ledger information for the item. A certification authority can include electrical circuitry configured to process computer-executable instructions. Although described herein primarily with respect to digital technology, a certification authority may also include primarily analog components. For example, some or all of the distributed ledger and certification algorithms described herein may be implemented in analog circuitry or mixed analog and digital circuitry. A computing environment can include a specialized computer system based on a microprocessor, a mainframe computer, a digital signal processor, a portable computing device, a device controller, or a computational engine within an appliance, to name a few.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all the features and benefits set forth herein, as some features can be used or practiced separately from others. The scope of certain embodiments disclosed herein is indicated by the appended claims rather than by the foregoing description. All changes that come within the meaning and range of equivalency of the claims are to be embraced within their scope.

1. A device, comprising:
a wearable housing to distribute air to a nostril or a mouth;
an air filter to filter the air, wherein the air filter comprises a scented filter to dispense a selected scent;
a cover to cover the nostril or mouth;
one or more vital sign sensors coupled to the wearable housing and a transceiver to communicate vital sign with a remote computer with a 5G transceiver coupled to a processor to request remote processing of a task selected by the processor from one or more edge processors, and wherein the task includes AR task, VR task, machine recognition task, or 3D rendering task; and
a learning machine with a deep neural network wherein an input layer of the neural network comprises input neurons, outputs of the input layer are distributed to all neurons in a middle layer and outputs of the middle layer are distributed to all output states, and each output has transition probabilities to itself or to a next output, wherein the deep neural network is coupled to the one or more vital sign sensors and configured to detect an infectious illness affecting at least a portion of a population.

2. The device of claim 1, comprising vital sign sensors configured to be worn by people in the portion of the population, wherein the learning machine is configured to provide detection for the illness affecting people in the population.

3. The device of claim 1, wherein the wearable housing comprises one or more ducts coupleable to the wearable housing configured to deliver air at a predetermined flow rate to prevent outside air from entering the nostril or mouth during inhalation.

4. The device of claim 3, wherein the one or more ducts are configured to provide pressurized filtered air into the nostril or the mouth, wherein air pressure proximal the nostril or a mouth is greater than ambient air pressure.

5. The device of claim 1, wherein the wearable housing comprises an eyewear device.

6. The device of claim 1, comprising a head attachment with a processor and data storage and ducts or tubing connecting the air filter to the wearable housing.

7. The device of claim 1, comprising:
a head attachment connected to an eyewear;
a power scavenger to collect energy from an environment;
an energy storage device to receive scavenged energy; and
one or more sensors to detect air quality or presence of pathogen.

8. (canceled)

9. (canceled)

10. The device of claim 1, comprising an elongated nostril housing with a sensor, wherein the air filter and the sensor are positioned in the nostril housing.

11. The device of claim 1, comprising a sensor in the wearable housing to detect pathogen presence based on resistance, inductance or capacitance reading from the sensor.

12. The device of claim 1, wherein the wearable housing transmits sounds for bone conduction.

13. The device of claim 1, comprising an air valve coupled to the wearable housing to control air flow from breathing in or breathing out.

14. (canceled)

15. The device of claim 1, wherein the air filter is in the wearable housing.

16. The device of claim 1, comprising a humectant coated on the air filter to incapacitate a virus.

17-20. (canceled)
